(19) United States
(12) Patent Application Publication
(10) Pub. No.: US 2003/0186269 A1
(43) Pub. Date: Oct. 2, 2003

(54) Ssa-56 kda polypeptide and its fragments and polynucleotides encoding said polypeptides and therapeutic uses

(76) Inventors: Georges Bahr, Lille (FR); Cecile Cocude, Annoeullin (FR); Andre Capron, Phalempin (FR)

Correspondence Address: BURNS DOANE SWECKER & MATHIS L L P, POST OFFICE BOX 1404, ALEXANDRIA, VA, 22313-1404, US

(21) Appl. No.: 10/276,372

(22) PCT Filed: Mar. 12, 2001
(86) PCT No.: PCT/FR01/00725

(30) Foreign Application Priority Data
May 17, 2000 (FR) 00 06315

(51) Int. Cl. C12Q 1/68; G01N 33/53; C07H 21/04; A61K 38/17; C07K 14/47; C07K 16/18

(52) U.S. Cl. 435/6; 435/69.1; 435/7.1; 435/320.1; 435/325; 530/350; 530/388.1; 536/23.2; 514/12

(57) ABSTRACT

The invention concerns a novel SSA-56 kDA polypeptide and its fragments, cDNA cloning and polynucleotides encoding said polypeptides, cloning and/or expression vectors including said polynucleotides, cells transformed by said vectors and specific antibodies directed against said polypeptides. The invention also concerns methods for detecting and/or assaying said polypeptides and polynucleotides, corresponding diagnostic kits, a method for screening ligands, a method for detecting anti-Ro/SSA-like autoantibodies, a method for purifying a human biological fluid capable of containing anti-Ro/SSA-like autoantibodies, and compounds for use as medicines for preventing and/or treating viral pathologies such as AIDS and autoimmune diseases, in particular systemic lupus erythematosus (SLE)and Sjögren syndrome.

The present invention relates to a novel Ro/SSA-like polypeptide, newly named SSA-56 kDa, and to its fragments, to the cloning of the cDNA and to the polynucleotides encoding said polypeptides, to cloning and/or expression vectors which include said polynucleotides, to cells transformed with said vectors and to specific antibodies against polypeptides. The invention also relates to methods for detecting and/or assaying said polypeptides and polynucleotides, to the corresponding diagnostic kits, to a method for screening ligands, to a method for detecting anti-Ro/SSA-like autoantibodies, to a method for purifying a human biological fluid liable to contain anti-Ro/SSA-like autoantibodies, and to compounds which can be used as a medicinal product for preventing and/or treating viral diseases such as AIDS and autoimmune diseases, in particular systemic lupus erythematosus (SLE) and Sjögren's syndrome.

Muramyl peptides are, among synthetic immunomodulators, those which have shown a large number of immunopharmacological effects on cells of the monocyte/macrophage line, potentiating their nonspecific resistance to infection, increasing the tumoricidal activity of macrophages, and also acting as vaccine adjuvants. Murabutide (MB), an analog of muramyl dipeptide (MDP), has been selected for its particularly promising biological profile and its good tolerance in animals and in humans. Specifically, unlike MDP and many other analogs, it is demonstrated that MB is not pyrogenic, does not induce inflammatory reactions and has not shown any severe toxicity in clinical studies in healthy volunteers and patients suffering from cancer.

By virtue of its biological capacities, MB is an antiviral agent which is promising in the AIDS (acquired immunodeficiency syndrome) field. Specifically, MB inhibits human immunodeficiency virus (HIV) replication in macrophages and dendritic cells, but also in peripheral blood mononuclear cells (PBMCs) of infected patients. Thus, given these biological characteristics, the immunomodulator MB was the subject of a granted French patent No. FR 2 724 845 entitled “Compositions of muramyl peptides capable of inhibiting up to 100% the replication of an acquired immunodeficiency virus such as HIV”. In addition, phase I and phase IIa clinical trials carried out to the end on HIV+ patients have demonstrated good clinical tolerance of MB.

The inventors have demonstrated that MB exerts a strong inhibition of viral replication in the PBMCs of CD8 lymphocyte-depleted patients, activated with phytohemagglutinin (PHA) and cultured with interleukin 2 (IL-2). Specifically, MB inhibits by 70 to 100% the level of HIV viral protein p24 in the culture supernatants. This effect correlates with the level of expression of viral messenger RNAs (nonspliced and single-spliced). In addition, analysis of the profile of secreted cytokines and chemokines has demonstrated that MB induces reproduction of chemokines known to be inhibitors of HIV replication. However, this induction does not appear to correlate completely with the inhibitory effect of MB. The inhibition of HIV replication by MB does not only involve induction of β-chemokine production, since MB is also involved at the level of the proviral DNA and of viral transcription. The lack of toxicity of MB in these same cell cultures has been verified by the inventors, who noted that not only does the number of live cells remain unchanged at the start of culturing, but it also appears to increase at the end of culturing.

The results obtained by the inventors therefore suggest that MB induces the production of cytokines or other factors not identified to date, which have a suppressor activity on HIV replication.

In order to identify these new factors involved in regulating viral replication, the inventors have used the “Differential Display-RT-PCR” (DD-RT-PCR) methodology, which is based on 2 essential steps; a first step of reverse transcription (RT) of the total cellular RNA in order to obtain complementary DNAs of all the RNAs which have a poly A tail, and then a second step of amplification by polymerase chain reaction (PCR) using the cDNAs, which serve as a matrix, and various pairs of primers in the presence of a radiolabeled nucleotide. The PCR products are then separated on a gel by electrophoresis. The differentially amplified fragments are cut out from the gel, reamplified and then cloned and sequenced.

DD-RT-PCR, carried out using PBMCs from an HIV+ patient, has made it possible for the inventors to select more than 130 cDNA fragments differentially expressed after treatment with MB. These fragments were subcloned into vector pCR2.1 (Invitrogen), then sequenced by automatic sequencing (ABI Prism 377, Perkin-Elmer). The sequences were analyzed for homology searches using the databanks and the Basic Local Alignment Search Tool (Blast 2) server of the NCBI.

Using the DD-RT-PCR technology, the inventors have isolated a novel protein, named Ro/SSA-like, which exhibits a relative sequence identity with the Ro/SSA protein.

Two peptide families exist, comprising four different molecular forms, lymphocytic and erythrocytic 60 kDa Ro/SSA, lymphocytic 52 kDa Ro/SSA and erythrocytic 54 kDa Ro/SSA (for review see Sibilia (1998)). The 60 kDa and 52 kDa Ro/SSA polypeptides associate directly or indirectly with a single-stranded RNA molecule (hYRNA) to form a ribonucleoprotein complex. Another protein, called La/SSB is present in this complex. The 60 kDa Ro/SSA isoform has a single zinc finger motif which has cysteine residues capable of binding DNA and RNA, and a consensus ribonucleoprotein (RNP) binding motif (Lopez-Luna et al. (1995)). Thus, the 60 kDa Ro/SSA is capable of directly binding hyRNAs (Human Cytoplasmic RNAs). The 52 kDa Ro/SSA isoform has two zinc finger motifs and also a leucine zipper sequence which binds DNA and which allows protein-protein interactions which lead to intra-molecular dimerization. This isoform does not have a consensus ribonucleoprotein (RNP) binding motif and is thus incapable of binding hyRNA. Its binding to the complex is thought to occur via calreticulin. The erythrocytic 54 kDa Ro/SSA isoform isolated by Rader et al. (1989) has various epitopes common to 52 kDa Ro/SSA. The latter isoform has not been sequenced to date. The Ro/SSA ribonucleoprotein complex is present in most cell tissues (red blood cells, platelets), but the structure and amount thereof vary depending on the tissues, the species and the stage of embryonic development. Its function is unknown but its structure, which allows it to bind nucleic acids, in particular RNA, and the existence of homologies with certain proteins for genomic regulation suggest that it contributes to the mechanisms of DNA transcription. Sera from patients suffering from autoimmune diseases, such as systemic lupus erythematosus (SLE) and neonatal lupus erythematosus, and from Sjögren's syndrome often exhibit antibodies against normal Ro/SSA cellular proteins.

Autoimmune diseases are diseases of the immune system, characterized by the production of antibodies (called autoantibodies) which react with antigens (called autoantigens) originating from the tissues of the patient himself or herself (for review see Schwartz et al. (1984)). The autoimmune diseases of the present invention comprise, nonexhaustively, the following diseases: uveitis, Bechet's disease, sarcoidosis, Sjögren's syndrome, rheumatoid arthritis, juvenile arthritis, Fiessinger-Leroy-Reiter syndrome, gout, osteoarthrosis, systemic lupus erythematosus, acute disseminated lupus erythematosus, polymyositis, myocarditis, primary biliary cirrhosis, Crohn's disease, ulcerative colitis, multiple sclerosis and other demyelinating diseases, aplastic anemia, essential thrombocytopenic purpura, multiple myeloma and B-lymphocyte lymphoma, Simmonds' disease panhypo-pituitarism, Basedow-Graves' disease and Graves' ophthalmopathy, subacute thyroiditis and Hashimoto's disease, Addison's disease, and insulin-dependent diabetes mellitus (type 1). More particularly, the autoimmune diseases of the invention correspond to SLE or to Sjögren's syndrome, or to chronic viral diseases exhibiting clinical manifestations similar to autoimmune diseases, such as AIDS and hepatitis C. These diseases can be subdivided into organ-specific diseases and systemic diseases. The organ-specific diseases affect only one organ, such as the thyroid gland, or only one physiological system, such as the neuro-muscular system. The autoantigens involved in organ-specific diseases are, first, organ-specific antigens and may be involved in the pathology of the disease. For example, autoantibodies against thyroglobulin are observed in autoimmune thyroiditis and thyroglobulin appears to be involved in the pathology of the disease. Systemic autoimmune diseases, on the other hand, affect many physiological systems. The autoantibodies involved in systemic autoimmune diseases generally react with more ubiquitous autoantigens, which include a group of antigens present in the cell nucleus. The latter group includes DNA, histones and a large number of ribonucleoproteins. Autoimmune diseases exhibit a wide variety of clinical signs and symptoms; however, the production of circulating autoantibodies against ribonucleoproteins (RNPs) appears to be a characteristic common to rheumatic autoimmune diseases. The autoantigens most common in systemic lupus erythematosus (SLE) and associated similar diseases are the ribonucleoproteins Ro/SSA, La/SSB, nRNP and Sm.

One of the objects of the present invention is to provide the Ro/SSA-like protein, to which serum antibodies produced by certain patients suffering from autoimmune diseases are capable of binding.

A subject of the present invention is therefore an isolated polypeptide, named Ro/SSA-like, of amino acid sequence SEQ ID No. 2. This sequence comprises conserved consensus domains which are readily identifiable by those skilled in the art. Among these conserved consensus domains, mention should be made of the sequence of amino acids 16 to 54 of the sequence SEQ ID No. 2, which comprises the zinc finger motif, the sequence of amino acids 91 to 123 of the sequence SEQ ID No. 2, which comprises a cysteine- and histidine-rich region, called B Box, and the sequence of amino acids 190 to 245 of the sequence SEQ ID No. 2, which comprises the leucine zipper motif. In the present invention, the polypeptide according to the invention will be indifferently named Ro/SSA-like or SSA-56.

The polypeptide according to the invention is characterized in that it is capable of binding to a nucleic acid sequence, and in that it comprises at least one nucleic acid binding domain selected from the group composed of a zinc finger domain and a leucine zipper domain.

The expression “binding to a DNA sequence” is intended to denote a specific interaction between the polypeptide of the invention and a DNA sequence by means of a series of weak bonds formed between the amino acids of the protein and the bases. The polypeptide according to the invention has at least one DNA binding domain which contains at least one of the known protein motifs capable of interacting with DNA, i.e. the zinc finger structure, the helix-turn-helix structure, the helix-loop-helix structure and the leucine zipper.

The term “zinc finger motif” is intended to denote a sequence of about twenty amino acids which, in space, are in the shape of a finger. Two types thereof exist: those which contain four cysteines (C4) and those which contain two cysteines and two histidines (C2H2). These amino acids define the nature of the finger and are located at its base, and a Zn++ ion is located at the center of the square formed by these four amino acids.

The expression “motif of the leucine zipper type” is intended to denote motifs preferably belonging to dimeric transcription factors which are either homodimers or heterodimers. The monomer consists of a sequence which is basic in nature and which interacts specifically with nucleic acid, preferably DNA, and of an α-helical hydrophobic domain which interacts with the homologous domain of the other chain. In this domain, there is one leucine every 7 amino acids, i.e. at each turn of the helix. All these leucines are aligned and interaction between the two monomers occurs at the level thereof. The polypeptide according to the invention has a motif of the leucine zipper type.

The isolated polypeptide is characterized in that it comprises a polypeptide chosen from:

a) a polypeptide of sequence SEQ ID No. 2;

b) a variant polypeptide of a polypeptide of amino acid sequence defined in a);

c) a polypeptide homologous to the polypeptide defined in a) or b) and comprising at least 80%, preferably at least 85%, 87%, 90%, 95%, 97%, 98%, 99%, identity with said polypeptide of a);

d) a fragment of at least 15 consecutive amino acids, preferably of at least 17, 20, 23, 25, 30, 40, 50, 100 consecutive amino acids, of a polypeptide defined in a), b) or c), with the exception of the fragment of sequence SEQ ID No. 4;

e) a biologically active fragment of a polypeptide defined in a), b) or c), with the exception of the fragment of sequence SEQ ID No. 4.

In the present description, the term polypeptide will be used to denote a protein or a peptide equally.

The term “variant polypeptide” will be intended to mean all the mutated polypeptides which may exist naturally, in particular in humans, and which correspond especially to truncations, substitutions, deletions and/or additions of amino acid residues.

The term “homologous polypeptide” will be intended to denote the polypeptides having, compared with the natural Ro/SSA-like polypeptide, certain modifications, such as in particular a deletion, addition or substitution of at least one amino acid, a truncation, an extension and/or a chimeric fusion. Among the homologous polypeptides, preference is given to those the amino acid sequence of which exhibits at least 80% identity, preferably at least 85%, 87%, 90%, 93%, 95%, 97%, 98%, 99% identity, with the amino acid sequences of the polypeptides according to the invention. In the case of a substitution, one or more consecutive or nonconsecutive amino acids can be replaced with “equivalent” amino acids. The expression “equivalent” amino acid is herein intended to denote any amino acid which can be substituted for one of the amino acids of the basic structure without, however, modifying the essential functional properties or characteristics, such as their biological activity, of the corresponding polypeptides, such as the in vivo induction of antibodies capable of recognizing the polypeptide the amino acid sequence of which is included in the amino acid sequence SEQ ID No. 2, or one of its fragments. These equivalent amino acids can be determined either based on their structural homology with the amino acids for which they substitute, or on the results of assays for cross biological activity which the various polypeptides are liable to produce. By way of example, mention will be made of the possibilities of substitutions which may be made without a profound modification of the biological activities of the corresponding modified polypeptides resulting therefrom; replacement, for example, of leucine with valine or isoleucine, of aspartic acid with glutamic acid, of glutamine with asparagine, of arginine with lysine, etc., it naturally being possible to envision the reverse substitutions under the same conditions.

The expression “biologically active fragment” will be intended to denote in particular a fragment of amino acid sequence of a polypeptide according to the invention having at least one of the structural characteristics of the polypeptide of the invention, i.e. a conserved domain of the zinc finger and/or leucine zipper and/or B Box type, or functional properties of the polypeptide of the invention, in particular in that it comprises a DNA and/or RNA nucleic acid binding activity. The variant polypeptide, the homologous polypeptide or the polypeptide fragment according to the invention has at least 10%, preferably at least 20%, 30%, 40%, 50%, 60%, 70%, 80%, 90%, 95%, of the nucleic acid binding activity. Various protocols have been described and are accessible to those skilled in the art for demonstrating the ability of polypeptides to bind nucleic acids. The examples below propose biological functions for the Ro/SSA-like protein according to the peptide domains of this protein and thus make it possible for those skilled in the art to identify the biologically active fragments.

The term “polypeptide fragment” is intended to denote a polypeptide comprising a minimum of 15 consecutive amino acids, preferably at least 17, 20, 23, 25, 30, 40, 50, 100, consecutive amino acids. The polypeptide fragments according to the invention obtained by cleaving said polypeptide with a proteolytic enzyme or with a chemical reagent, or else by placing said polypeptide in a very acidic environment, are also part of the invention.

Preferably, a polypeptide according to the invention is a polypeptide consisting of the sequence SEQ ID No. 2 or of the sequence having at least 80% identity, preferably at least 85%, 90%, 95%, 98% and 99% identity, with SEQ ID No. 2 after optimal alignment. The expression “polypeptide the amino acid sequence of which exhibits a percentage identity of at least 80%, preferably of at least 85%, 90%, 95%, 98% and 99%, after optimal alignment, with a reference sequence” is intended to denote the polypeptides having certain modifications compared with the reference polypeptide, such as in particular one or more deletions or truncations, an extension, a chimeric fusion and/or one or more substitutions.

Among the polypeptides the amino acid sequence of which exhibits a percentage identity of at least 80%, preferably of at least 85%, 90%, 95%, 98% and 99%, after optimal alignment, with the sequences SEQ ID No. 2 or with one of their fragments according to the invention, preference is given to the variant polypeptides encoded by the variant peptide sequences as defined above, in particular the polypeptides the amino acid sequence of which has at least one mutation corresponding in particular to a truncation, deletion, substitution and/or addition of at least one amino acid residue compared with the sequences SEQ ID No. 2 or with one of their fragments, more preferably the variant polypeptides having a mutation associated with a pathology.

The invention also relates to a purified or isolated polynucleotide, characterized in that it encodes a polypeptide as defined above. Preferably, the polynucleotide according to the invention has the sequence SEQ ID No. 1.

The purified or isolated polynucleotide according to the invention is characterized in that it comprises a polynucleotide chosen from:

a) a polynucleotide of sequence SEQ ID No. 1;

b) a fragment of at least 15 consecutive nucleotides, preferably of at least 18, 21, 24, 27, 30, 35, 40, 50, 75, 100 consecutive nucleotides, of the sequence SEQ ID No. 1, with the exception of the polynucleotide of sequence SEQ ID No. 3 and of the polynucleotides of sequences AK001231 and N46696 of the EMBL databank, and of the polynucleotide of sequence SEQ ID No. 5505 of patent application EP 0 679 716;

c) a nucleic acid sequence exhibiting a percentage identity of at least 85%, preferably of at least 90%, 95%, 98% and 99%, after optimal alignment, with a sequence defined in a) or b);

d) the complementary sequence or the RNA sequence corresponding to a sequence as defined in a), b) or c).

The terms “nucleic acid”, “nucleic acid sequence”, “polynucleotide”, “oligonucleotide”, “polynucleotide sequence” and “nucleotide sequence”, all terms which will be used equally in the present description, are intended to denote a precise series of nucleotides, which may or may not be modified, making it possible to define a fragment or a region of a nucleic acid, which may or may not comprise unnatural nucleotides, and which may correspond equally to a double-stranded DNA, a single-stranded DNA and transcription products of said DNAs, and/or an RNA fragment.

It should be understood that the present invention does not relate to the nucleotide sequences in their natural chromosomal environment, that is to say in the natural state. They are sequences which have been isolated and/or purified, that is to say they have been taken directly or indirectly, for example by copying, their environment having been at least partially modified. Thus, nucleic acids obtained by chemical synthesis are also intended to be denoted.

The expression “polynucleotide of complementary sequence” is intended to denote any DNA the nucleotides of which are complementary to those of SEQ ID No. 1, or of a portion of SEQ ID No. 1, and the orientation of which is reversed.

For the purpose of the present invention, the term “percentage identity” between two nucleic acid or amino acid sequences is intended to denote a percentage of nucleotides or of amino acid residues which are identical between the two sequences to be compared, obtained after the best alignment, this percentage being purely statistical and the differences between the two sequences being distributed randomly and over their entire length. The term “best alignment” or “optimal alignment” is intended to denote the alignment for which the percentage identity determined as below is highest. The sequence comparisons between two nucleic acid or amino acid sequences are conventionally carried out by comparing these sequences after having aligned them optimally, said comparison being carried out by segment or by “window of comparison” so as to identify and compare the local regions of sequence similarity. The optimal alignment of the sequences for the comparison may be carried out, besides manually, by means of the local homology algorithm of Smith and Waterman (1981), by means of the local homology algorithm of Neddleman and Wunsch (1970), by means of the similarity search method of Pearson and Lipman (1988), by means of computer programs using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimum alignment, the BLAST program is preferably used, with the BLOSUM 62 matrix. The PAM or PAM250 matrices may also be used.

The percentage identity between two nucleic acid or amino acid sequences is determined by comparing these two sequences aligned optimally, the nucleic acid or amino acid sequence to be compared possibly comprising additions or deletions with respect to the reference sequence for optimal alignment between these two sequences. The percentage identity is calculated by determining the number of identical positions for which the nucleotide or the amino acid residue is identical between the two sequences, dividing this number of identical positions by the total number of positions compared and multiplying the result obtained by 100 so as to obtain the percentage identity between these two sequences.

The expression “nucleic acid sequences exhibiting a percentage identity of at least 85%, preferably of at least 90%, 95%, 98% and 99%, after optimal alignment, with a reference sequence” is intended to denote the nucleic acid sequences which, compared to the reference nucleic acid sequence, have certain modifications, such as in particular a deletion, a truncation, an extension, a chimeric fusion and/or a substitution, in particular of the point type, and the nucleic acid sequence of which exhibits at least 85%, preferably at least 90%, 95%, 98% and 99%, identity, after optimal alignment, with the reference nucleic acid sequence. They are preferably sequences whose complementary sequences are capable of hybridizing specifically with the sequences SEQ ID No. 1 of the invention. Preferably, the specific or high stringency hybridization conditions will be such that they ensure at least 85%, preferably at least 90%, 95%, 98% and 99%, identity, after optimal alignment, between one of the two sequences and the sequence complementary to the other.

Hybridization under high stringency conditions means that the conditions of temperature and of ionic strength are chosen such that they allow the hybridization between two complementary DNA fragments to be maintained. By way of illustration, high stringency conditions for the hybridization step for the purposes of defining the polynucleotide fragments described above are advantageously as follows:

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10×Denhardt's, 5% of dextran sulfate and 1% of salmon sperm DNA; (2) hybridization per se for 20 hours at a temperature which depends on the length of the probe (i.e.: 42° C. for a probe >100 nucleotides in length), followed by 2 washes of 20 minutes at 20° C. in 2×SSC+2% SDS and 1 wash of 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length may be adjusted by those skilled in the art for longer or shorter oligonucleotides, according to the teaching of Sambrook et al., 1989.

Among the nucleic acid sequences exhibiting a percentage identity of at least 85%, preferably of at least 90%, 95%, 98% and 99%, after optimal alignment, with the sequence according to the invention, preference is also given to the nucleic acid sequences which are variants of SEQ ID No. 1, or of their fragments, that is to say all the nucleic acid sequences corresponding to allelic variants, that is to say individual variations of the sequences SEQ ID No. 1. These natural mutated sequences correspond to polymorphisms present in mammals, in particular in humans, and especially to polymorphisms which may lead to the occurrence of a pathology.

The expression “variant nucleic acid sequence” is also intended to denote any RNA or cDNA resulting from a mutation and/or variation of a splice site of the genomic nucleic acid sequence the cDNA of which has the sequence SEQ ID No. 1.

More particularly, the invention relates to a purified or isolated nucleic acid according to the present invention, characterized in that it comprises or consists of one of the sequences SEQ ID No. 1, of the sequences complementary thereto, or of the RNA sequences corresponding to SEQ ID No. 1. The primers or probes, characterized in that they comprise a nucleic acid sequence according to the invention, are also part of the invention. Thus, the present invention for detecting, identifying, assaying or amplifying a nucleic acid sequence also relates to the primers or the probes according to the invention which may make it possible in particular to demonstrate or to distinguish the variant nucleic acid sequences, or to identify the genomic sequence of the genes the cDNA of which is represented by SEQ ID No. 1, in particular using an amplification method such as the PCR method or a related method. According to the invention, the polynucleotides which can be used as a probe or as a primer in methods for detecting, identifying, assaying or amplifying a nucleic acid sequence are a minimum of 15 bases, preferably at least 18, 20, 25, 30, 40, 50 bases, in length.

The polynucleotides according to the invention may thus be used as a primer and/or probe in methods using in particular the PCR (polymerase chain reaction) technique (Rolfs et al., 1991). This technique requires choosing pairs of oligonucleotide primers bordering the fragment which must be amplified. Reference may, for example, be made to the technique described in U.S. Pat. No. 4 683 202. The amplified fragments can be identified, for example after agarose or polyacrylamide gel electrophoresis, or after a chromatographic technique such as gel filtration or ion exchange chromatography, and then sequenced. The specificity of the amplification can be controlled using, as primers, the nucleotide sequences of polynucleotides of the invention and, as matrices, plasmids containing these sequences or else the derived amplification products. The amplified nucleotide fragments may be used as reagents in hybridization reactions in order to demonstrate the presence, in a biological sample, of a target nucleic acid of sequence complementary to that of said amplified nucleotide fragments. The invention is also directed toward the nucleic acids which can be obtained by amplification using primers according to the invention.

Other techniques for amplifying the target nucleic acid may advantageously be employed as an alternative to PCR (PCR-like) using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, they involve amplifying the DNA with a polymerase; when the sample of origin is an RNA, a reverse transcription should be carried out beforehand. A large number of methods currently exist for this amplification, such as, for example, the SDA (strand displacement amplification) technique (Walker et al., 1992), the TAS (transcription-based amplification system) technique described by Kwoh et al. (1989), the 3SR (self-sustained sequence replication) technique described by Guatelli et al. (1990), the NASBA (nucleic acid sequence based amplification) technique described by Kievitis et al. (1991), the TMA (transcription mediated amplification) technique, the LCR (ligase chain reaction) technique described by Landegren et al. (1988), the RCR (repair chain reaction) technique described by Segev (1992), the CPR (cycling probe reaction) technique described by Duck et al. (1990), and the Q-beta-replicase amplification technique described by Miele et al. (1983). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA obtained in the biological sample. The cDNA obtained will then serve as a target for the primers or the probes used in the amplification or detection method according to the invention.

The probe hybridization technique may be carried out in various ways (Matthews et al., 1988). The most general method consists in immobilizing the nucleic acid extracted from the cells of various tissues or from cells in culture, on a support (such as nitrocellulose, nylon or polystyrene), so as to produce, for example, DNA chips, and then in incubating the immobilized target nucleic acid with the probe, under well-defined conditions. After hybridization, the excess probe is removed and the hybrid molecules formed are detected using the appropriate method (measuring the radioactivity, the fluorescence or the enzymatic activity linked to the probe).

According to another embodiment of the nucleic acid probes according to the invention, the latter may be used as capture probes. In this case, a probe, termed “capture probe”, is immobilized on a support and is used to capture, by specific hybridization, the target nucleic acid obtained from the biological sample to be tested, and the target nucleic acid is then detected using a second probe, termed “detection probe”, labeled with a readily detectable element.

Among the advantageous nucleic acid fragments, mention should, moreover, be made in particular of antisense oligonucleotides, i.e. oligonucleotides the structure of which ensures, by hybridization with the target sequence, inhibition of expression of the corresponding product. Mention should also be made of sense oligonucleotides which, by interacting with proteins involved in regulating the expression of the corresponding product, will induce either inhibition or activation of this expression. The oligonucleotides according to the invention are a minimum of 9 bases, preferably at least 10, 12, 15, 17, 20, 25, 30, 40, 50 bases, in length.

The probes, primers and oligonucleotides according to the invention may be labeled directly or indirectly with a radioactive or nonradioactive compound, by methods well known to those skilled in the art, in order to obtain a detectable and/or quantifiable signal. The polynucleotide sequences according to the invention which are unlabeled may be used directly as a probe or primer.

The sequences are generally labeled so as to obtain sequences which can be used for many applications. The primers or probes according to the invention are labeled with radioactive elements or with nonradioactive molecules. Among the radioactive isotopes used, mention may be made of ³²P, ³³P, ³⁵S, ³H or ¹²⁵I. The nonradioactive entities are selected from ligands such as biotin, avidin, streptavidin or dioxygenin, haptens, dyes and luminescent agents, such as radioluminescent, chemiluminescent, bioluminescent, fluorescent or phosphorescent agents.

The present invention also relates to the cloning and/or expression vectors comprising a nucleic acid or encoding a polypeptide according to the invention. Such a vector may also contain the elements required for the expression and, optionally, the secretion of the polypeptide in a host cell. Such a host cell is also a subject of the invention.

According to another aspect, the invention relates to an antisense expression vector. Such an expression vector contains a polynucleotide sequence according to the invention, inserted in reverse orientation into the expression vector. Thus, those skilled in the art readily recognize that an mRNA corresponding to the DNA in the antisense vector hybridizes with an mRNA corresponding to DNA in the sense vector. An antisense expression vector is a vector which expresses an antisense RNA of interest in a suitable host cell, either constitutively or after induction. The term “antisense” refers to any composition containing a specific nucleic acid sequence. The antisense molecules can be produced by methods such as synthesis or transcription. When such molecules are introduced into the cell, the complementary nucleotides combine with the natural sequences produced by the cell to form duplexes and thus block either the transcription or the translation of the polypeptide according to the invention. It also falls within the scope of the invention to produce antisense molecules capable of pairing with the RNA molecule with which the Ro/SSA-like protein is capable of associating to form a ribonucleoprotein.

Said vectors preferably comprise a promoter, translation initiation and termination signals, and also regions suitable for regulating transcription. It should be possible for them to be maintained stably in the cell and they may optionally possess particular signals specifying secretion of the translated protein. According to a particular embodiment of the invention, the promoter may be the promoter naturally present upstream of the gene encoding human Rb/SSA-like of the invention.

The various control signals are chosen as a function of the cellular host used. To this effect, the nucleic acid sequences according to the invention may be inserted into vectors which replicate autonomously in the chosen host, or vectors which integrate in the chosen host.

Among the systems which replicate autonomously, use is preferably made, depending on the host cell, of systems of the “plasmid”, “cosmid” or “minichromosome” type or systems of the viral type, the viral vectors possibly being in particular adenoviruses (Perricaudet et al., 1992), retroviruses, lentiviruses, poxviruses or herpesviruses (Epstein et al., 1992). Those skilled in the art are aware of the technology which can be used for each of these systems.

When integration of the sequence into the chromosomes of the host cell is desired, use may be made, for example, of systems of the plasmid or viral type; such viruses are, for example, retroviruses (Temin, 1986) or AAVs (Carter, 1993).

Among the nonviral vectors, preference is given to naked polynucleotides such as naked DNA or naked RNA according to the technique developed by the company VICAL, bacterial artificial chromosomes (BACs), yeast artificial chromosomes (YACs) for expression in yeast, mouse artificial chromosome (MACs) for expression in murine cells and, preferably, human artificial chromosomes (HACs) for expression in human cells,

Such vectors are prepared according to the methods commonly used by those skilled in the art, and the clones resulting therefrom can be introduced into a suitable host using standard methods, such as, for example, lipofection, electroporation, heat shock, transformation after chemical permeabilization of the membrane, or cell fusion.

The invention also comprises the host cells, in particular the eukaryotic and prokaryotic cells, transformed by the vectors according to the invention. Among the cells which can be used for the purposes of the present invention, mention may be made of bacterial cells (Olins and Lee, 1993), but also yeast cells (Buckholz, 1993), as well as animal cells, in particular mammalian cell cultures (Edwards and Aruffo, 1993), and in particular Chinese hamster ovary (CHO) cells. Mention may also be made of insect cells in which it is possible to use methods employing, for example, baculoviruses (Luckow, 1993). A preferred cellular host for expressing the proteins of the invention consists of Cos cells and Hela cells. According to a preferred embodiment, the host cell is transformed with an expression vector which allows the expression and/or, optionally, the secretion of the polypeptide according to the invention.

The invention also comprises the transgenic animals, preferably the mammals, except humans, comprising one of said transformed cells according to the invention. These animals can be used as models, for studying the etiology of a pathology associated with a deleterious modification of the animal homologue of the natural human Ro/SSA-like protein, or for studying the effects of a viral infection caused by an RNA virus, such as HIV, on the expression of the Ro/SSA-like protein in the presence or absence of an antiviral treatment, such as murabutide.

Among the mammals according to the invention, animals such as rodents, in particular mice, rats or rabbits, expressing a polypeptide according to the invention, are preferred.

The transgenic animals according to the invention can overexpress the genes encoding the protein according to the invention, or their homologous gene, or express said gene into which a mutation is introduced. These transgenic animals, in particular mice, are obtained, for example, by transfection of a copy of this gene under the control of a promoter which is strong and ubiquitous, or selective for a tissue type, or after viral transcription.

Alternatively, the transgenic animals according to the invention may be made deficient for the gene encoding the polypeptide of sequence SEQ ID No. 2, or their homologous genes, by targeted inactivation by homologous recombination possibly using the LOX-P/CRE recombinase system (Rohlmann et al., 1996) or by any other system for inactivating the expression of this gene. These transgenic animals are obtained, for example, by homologous recombination on embryonic stem cells, transfer of these stem cells to embryos, selection of the chimeras affected in the reproductive lines, and growth of said chimeras.

The cells or mammals transformed as described above can also be used as models in order to study the interactions between the polypeptides according to the invention and the chemical or protein compounds involved directly or indirectly in the activities of the polypeptides according to the invention, this being in order to study the various mechanisms and interactions involved. They may in particular be used to select products which interact with the polypeptides according to the invention, in particular the protein of SEQ ID No. 2, or their variants according to the invention, as a cofactor or as an inhibitor, in particular a competitive inhibitor, or which have an agonist or antagonist activity with respect to the activity of the polypeptides according to the invention. Preferably, said transformed cells or transgenic animals are used as a model, in particular for selecting products for combating pathologies associated with abnormal expression of this gene.

In addition to their use as an analytical model, the cells and mammals according to the invention can be used in a method for producing a polypeptide according to the invention, as described below.

The method for producing a polypeptide of the invention in recombinant form, which is itself included in the present invention, is characterized in that the transformed cells, in particular the cells of the present invention, are cultured under conditions which allow the expression and, optionally, the secretion of a recombinant polypeptide encoded by a nucleic acid sequence according to the invention, and in that said recombinant polypeptide is recovered. The recombinant polypeptides which can be obtained using this method of production are also part of the invention. They may be in glycosylated or unglycosylated form and may or may not have the tertiary structure of the natural protein.

The sequences of the recombinant polypeptides can also be modified in order to improve their solubility, in particular in aqueous solvents. Such modifications are known to those skilled in the art, such as, for example, the deletion of hydrophobic domains or the substitution of hydrophobic amino acids with hydrophilic amino acids.

These polypeptides can be produced from the nucleic acid sequences defined above, according to the techniques for producing recombinant polypeptides known to those skilled in the art. In this case, the nucleic acid sequence used is placed under the control of signals which allow its expression in a cellular host.

An effective system for producing a recombinant polypeptide requires having a vector and a host cell according to the invention. These cells can be obtained by introducing into host cells a nucleotide sequence inserted into a vector as defined above, and then culturing said cells under conditions which allow the replication and/or expression of the transfected nucleotide sequence.

The methods used for purifying a recombinant polypeptide are known to those skilled in the art. The recombinant polypeptide can be purified from cell lysates and extracts or from the culture medium supernatant, by methods used individually or in combination, such as fractionation, chromatography methods, and/or immunoaffinity techniques using specific monoclonal or polyclonal antibodies.

A preferred variant consists in producing a recombinant polypeptide fused to a “carrier” protein (chimeric protein). The advantage of this system is that it allows stabilization and a decrease in proteolysis of the recombinant product, an increase in the solubility during renaturation in vitro and/or simplification of the purification when the fusion partner has affinity for a specific ligand.

The polypeptides according to the present invention can also be obtained by chemical synthesis using one of the many known forms of peptide synthesis, for example techniques using solid phases (see in particular Stewart et al., 1984) or techniques using partial solid phases, by fragment condensation or by conventional synthesis in solution. The polypeptides obtained by chemical synthesis and which may comprise corresponding unnatural amino acids are also included in the invention.

The invention also relates to a monoclonal or polyclonal antibody and its fragments, characterized in that they selectively and/or specifically bind a polypeptide according to the invention. The chimeric antibodies, the humanized antibodies and the single-chain antibodies are also part of the invention. The antibody fragments according to the invention are preferably Fab, F(ab′)2, Fv or Fc fragments.

The polypeptides according to the invention make it possible to prepare monoclonal or polyclonal antibodies. The monoclonal antibodies may advantageously be prepared from hybridomas according to the technique described by Kohler and Milstein in 1975 and 1976.

The polyclonal antibodies can be prepared, for example, by immunizing an animal, in particular a mouse, with a polypeptide according to the invention combined with an adjuvant of the immune response, and then purifying the specific antibodies contained in the serum of the immunized animals, on an affinity column to which the polypeptide which served as antigen has been attached beforehand. The polyclonal antibodies according to the invention can also be prepared by purification on an affinity column on which a polypeptide according to the invention has been immobilized beforehand.

The polyclonal antisera and/or monoclonal antibodies of the invention, and also the autoantibodies of patients suffering from autoimmune diseases, directed against the Ro/SSA-like protein, can be used to analyze the structure and the function of the Ro/SSA-like protein and its fragments. According to a particular embodiment of the invention, the monoclonal or polyclonal antibody or the autoantibody is capable of inhibiting the interaction between the Ro/SSA-like polypeptides of the invention and the nucleic acid sequence to which these polypeptides bind, in order to impair the physiological function of said polypeptides according to the invention.

Idiotypes common to various autoantibodies from patients or an idiotype of the antibody of the invention can be used to generate anti-idiotype antibodies and their fragments. Specifically, the idiotypic (antigen-binding) structure of the antibody is antigenic and can therefore make it possible to produce specific antibodies against the idiotypic structure. Such anti-idiotypic antibodies are also one of the subjects of the present invention. The anti-idiotype antibody according to the invention may be capable of replacing the original antigen for some or all of the functions, the use and the properties of the original polypeptide of the invention; it may in particular be of use for blocking the binding of the anti-Ro/SSA-like antibodies to the native Ro/SSA-like protein in vivo, or for replacing the anti-Ro/SSA-like polypeptides of the invention in the methods described below which involve plasmaphoresis and extracorporeal immunoabsorption.

The invention also relates to methods for detecting and/or purifying a polypeptide according to the invention, characterized in that they use an antibody according to the invention. The invention also comprises purified polypeptides, characterized in that they are obtained using a method according to the invention.

Moreover, besides their use for purifying the polypeptides, the antibodies of the invention, in particular the monoclonal antibodies, may also be used for detecting these polypeptides in a biological sample. For these various uses, the antibodies of the invention may also be labeled in the same way as described previously for the nucleic acid probes of the invention, and preferably with labeling of the enzymatic, fluorescent or radioactive type.

The antibodies of the invention also constitute a means for analyzing the expression of a polypeptide according to the invention, for example using immunofluorescence, gold labeling and/or enzymatic immunoconjugates. More generally, the antibodies of the invention may advantageously be used in any situation where the expression of a polypeptide according to the invention must be observed, and more particularly in immunocytochemistry, in immunohistochemistry or in Western blotting experiments.

They may in particular make it possible to demonstrate abnormal expression of these polypeptides in biological specimens or tissues.

More generally, the antibodies of the invention may advantageously be used in any situation where the expression of a polypeptide according to the invention, which may be normal or mutated, must be observed. Thus, a method for detecting and/or assaying a polypeptide according to the invention, in a biological sample, comprising the steps of bringing the biological sample into contact with an antibody according to the invention and demonstrating the antigen-antibody complex formed, is also a subject of the invention.

Also falling within the context of the invention is a kit of reagents for detecting and/or assaying a polypeptide according to the invention, in a biological sample, characterized in that it comprises the following elements: (i) a monoclonal or polyclonal antibody as described above; (ii) where appropriate, the reagents for constituting the medium suitable for the immunoreaction; (iii) where appropriate, the reagents for detecting the antigen-antibody complexes produced by the immunoreaction. This kit is in particular of use for carrying out Western blotting experiments; these experiments make it possible to study the regulation of expression of the polypeptide according to the invention using tissues or cells. This kit is also of use in immunoprecipitation experiments for demonstrating in particular the proteins which interact with the polypeptide according to the invention. This kit is also of use for detecting and/or assaying a polypeptide according to the invention using a method which involves the ELISA technique, immunofluorescence, radioimmunology (RIA technique) or an equivalent technique.

The invention also comprises a method for detecting and/or assaying a polynucleotide according to the invention, in a biological sample, characterized in that it comprises the following steps: (i) isolating the DNA from the biological sample to be analyzed, or obtaining a cDNA from the RNA of the biological sample; (ii) specifically amplifying the DNA encoding the polypeptide according to the invention using primers; (iii) analyzing the amplification products. An object of the invention is also to provide a kit for detecting and/or assaying a nucleic acid according to the invention, in a biological sample, characterized in that it comprises the following elements: (i) a pair of nucleic acid primers according to the invention, (ii) the reagents required to carry out a DNA amplification reaction and, optionally, (iii) a component for verifying the sequence of the amplified fragment, more particularly a probe according to the invention.

The invention also comprises a method for detecting and/or assaying nucleic acid according to the invention, in a biological sample, characterized in that it comprises the following steps: (i) of bringing a polynucleotide according to the invention into contact with a biological sample; (ii) of detecting and/or assaying the hybrid formed between said polynucleotide and the nucleic acid of the biological sample.

Thus, the appropriate polynucleotide sequence can be used in in situ hybridization reactions in order to detect the level of expression of genes encoding the Ro/SSA-like antigen against which the patient's autoantibodies are directed, in specific tissues or in PBMCs. The level of gene expression can thus be quantified in the patients and compared with healthy controls, or can be compared between various tissues.

An object of the invention is therefore to provide a kit for detecting and/or assaying a nucleic acid according to the invention, in a biological sample, characterized in that it comprises the following elements: (i) a probe according to the invention, (ii) where appropriate, the reagents required to carry out a hybridization reaction, and/or, where appropriate, (iii) a pair of primers according to the invention and also the reagents required for a DNA amplification reaction.

The methods for determining an allelic variability, a mutation, a deletion, a loss of heterozygocity or any genetic abnormality of the gene encoding the polypeptide according to the invention, characterized in that they use a nucleic acid sequence, a polypeptide or an antibody according to the invention, are also part of the invention.

These mutations can be detected directly by analyzing the nucleic acid and the sequences according to the invention (RNA or cDNA), but also via the polypeptides according to the invention. In particular, the use of an antibody according to the invention which recognizes an epitope carrying a mutation makes it possible to distinguish between a “healthy” protein and a protein “associated with a pathology”.

This method of diagnosis and/or of prognostic assessment can be used preventively, or so as to serve in establishing and/or confirming a clinical condition in a patient. The analysis can be carried out by sequencing all or part of the gene (i.e. the exons), or by other methods known to those skilled in the art. It is in particular possible to use methods based on PCR, for example PCR-SSCP, which makes it possible to detect point mutations. The analysis can also be carried out by attachment of a probe according to the invention to a DNA chip containing at least one polynucleotide according to the invention, and hybridization on these microplates. A DNA chip containing a sequence according to the invention is also one of the subjects of the invention.

Similarly, a protein chip containing an amino acid sequence according to the invention is also a subject of the invention. Such a protein chip makes it possible to study the interactions between the polypeptides according to the invention and other proteins or chemical compounds, and may thus be of use in screening compounds which interact with the polypeptides according to the invention. The protein chips according to the invention can also be used for detecting the presence of antibodies against the polypeptides according to the invention in the serum of patients. A protein chip containing a monoclonal or polyclonal antibody, or an anti-idiotype antibody, or their fragments, according to the invention may also be used.

The invention also relates to a method for screening ligands capable of affecting the transcription in vitro and/or in vivo of the gene naturally encoding the polypeptide of the invention, which comprises the following steps: (i) bringing a cell chosen from the host cell of the invention and eukaryotic cells, preferably a human cell, expressing the polypeptide of the invention, into contact with one or more potential ligands, in the presence of reagents required to carry out a transcription reaction, and (ii), detecting and/or measuring the transcriptional activity. The gene encoding the polypeptide according to the invention, which is present in the host cell or in a eukaryotic cell, preferably a human cell, corresponds at least to a polynucleotide sequence encoding the polypeptide of the invention, preferably in the form of genomic DNA or of cDNA, functionally linked to the promoter sequence of the human Ro/SSA-like gene or of the homologous gene of an animal species such as the mouse. DD-RT-PCR technology also constitutes a method for screening ligands according to the invention capable of affecting the transcription of the gene encoding the Ro/SSA-like polypeptide according to the invention.

The expression “ligand capable of affecting the transcription in vitro and/or in vivo of the gene naturally encoding the polypeptide of the invention” is intended to define all the compounds capable of interacting with the regulatory polynucleotide sequences (promoter, upstream sequence, enhancer, silencer, insulator, etc.) of the gene naturally encoding the polypeptide according to the invention or the compounds capable of interacting with transcription factors (general transcription factors or tissue-specific factors) involved in regulating the transcription of the gene encoding the polypeptide according to the invention, so as to form a complex capable of affecting the transcription of the gene encoding Ro/SSA-like of the invention, i.e. of increasing, of decreasing, of modulating or of eliminating the transcription of said gene. The ligands identified include proteins, nucleic acids, carbohydrates, lipids and all chemical molecules capable of affecting the transcription in vitro and/or in vivo of the gene naturally encoding the polypeptide of the invention.

The techniques for detecting and/or measuring transcriptional activity are known to those skilled in the art. Mention should in particular be made of Northern blotting technology and RT-PCR technology, which can be implemented with the polynucleotides of the invention used, respectively, as a probe or as a primer.

Preferably, the biological sample according to the invention in which the detecting, the assaying and/or the screening carried out consists of a body fluid, for example a human or animal serum, blood or urine, or of biopsies.

One of the objects of the invention is also to provide ligands which affect the transcription in vitro and/or in vivo of the gene naturally encoding the polypeptide of the invention and which can be obtained using the above screening method. Synthetic immunomodulators, such as the compounds of the muramyl peptide family, and more particularly murabutide (MB), constitute ligands according to the invention.

The present invention also relates to an agent for diagnosing human autoimmune diseases, characterized in that said diagnostic agent is selected from a polypeptide according to the invention, an anti-idiotypic antibody according to the invention, and a host cell according to the invention which is transformed with an expression vector capable of effectively expressing a polypeptide of the invention. According to a particular embodiment, the diagnostic agent according to the invention is characterized in that said polypeptide, said anti-idiotypic antibody and said anti-idiotypic antibody fragments are coupled to a solid support directly or indirectly via a spacer arm, and are optionally labeled directly or indirectly with a signal-generating label; this label is selected from radioactive isotopes and nonisotopic entities. The nonisotopic entities are selected from enzymes, dyes, haptens, luminescent agents, such as radioluminescent, chemiluminescent, bioluminescent, fluorescent or phosphorescent agents, and ligands such as biotin, avidin, streptavidin or digoxygenin.

The invention also relates to a diagnostic kit, characterized in that it contains a diagnostic agent as defined above.

The detecting and the titering of the autoantibodies are used to diagnose and to follow the progression of autoimmune diseases or chronic viral diseases exhibiting clinical manifestations similar to autoimmune diseases, such as AIDS and hepatitis C. In clinical laboratories, the test directed against anti-nuclear antibodies makes it possible to titer the presence of autoantibodies which react with nuclear autoantigens. This test is widely used to detect autoantibodies against nuclear antigens, and is used for the diagnosis of many autoimmune systemic diseases. The present invention therefore proposes to provide a method for detecting anti-Ro/SSA-like autoantibodies in a human biological fluid, in order to carry out a novel diagnostic test for many autoimmune diseases. The method for detecting anti-Ro/SSA-like autoantibodies in a human biological fluid comprises the steps (i) of bringing said biological fluid into contact with a diagnostic agent according to the invention, characterized in that said autoantibodies react with said diagnostic agent; and (ii) of demonstrating the autoantibody/polypeptide complex or the autoantibody/anti-idiotype antibody complex formed. The autoantibodies detected by the method are preferably associated with autoimmune diseases preferably selected from the group of systemic lupus erythematosus (SLE) and Sjögren's syndrome, from the group of chronic pathologies exhibiting autoimmune manifestations, such as AIDS or hepatitis B and C, and from the group of viral pathologies, and preferably those caused by an infection with an RNA virus. The autoantibodies detected by this method may also be present in the biological fluid of patients whose cells have undergone a stress. The term “stress” is intended to denote a physical, chemical or biological agent causing a reaction of the cell. Among physical agents, mention should be made, inter alia, of beta-rays, gamma-rays, X-rays, ultraviolet radiation, infrared radiation and visible light. In addition, culturing conditions, which may be aerobic or anaerobic, the pH of the culture medium, which may be acidic, basic or neutral, or the concentration of oxidizing agent (free radicals, etc.) or of another element in the cellular and/or extra-cellular environment are liable to constitute stress factors which are physical in nature. The term “chemical agent” is intended to denote any chemical compound capable of interacting with the cell or one of the cell membrane or intracellular components; for example, intercalating agents such as ethidium bromide or propidium iodide constitute chemical compounds according to the invention. The biological compounds of the invention correspond to all compounds capable of causing a biological cellular reaction. Mention may be made, nonexhaustively, of all the molecules which interact with a membrane-bound receptor, such as, for example, intracellular communication molecules, hormones, cytokines, lymphokines, interleukins or antibodies. Viruses also constitute biological agents according to the invention.

The invention also relates to a kit for carrying out the above method for detecting anti-Ro/SSA-like autoantibodies; this kit contains at least one diagnostic agent according to the invention.

One of the therapeutic uses of the polypeptides of the invention consists in using the expressed polypeptide of the invention to absorb the patient's circulating autoantibodies. Thus, the polypeptide according to the invention or the anti-idiotypic antibody according to the invention, or one of its fragments, can be bound to solid-phase particles which are brought into contact with the patient's biological fluid during, for example, plasma phoresis or extra-corporeal immunoabsorption, in order to reduce the circulating level of anti-Ro/SSA-like autoantibodies in the patient. The invention provides a method for purifying a human biological fluid liable to contain anti-Ro/SSA-like autoantibodies and comprising the steps: (i) of bringing said biological fluid into contact with a polypeptide according to the invention or an anti-idiotypic antibody according to the invention, or one of its fragments, under conditions which allow the formation of an autoantibody/polypeptide complex or of an autoantibody/anti-idiotype antibody complex formed; (ii) separating the biological fluid and the complex formed in step (i); and (iii) recovering the biological fluid obtained in step (ii). The human biological fluid thus purified and able to be obtained using the above method can be used to prepare a composition intended for the therapeutic treatment of patients suffering from autoimmune diseases, preferably selected from the group of systemic lupus erythematosus (SLE) and Sjögren's syndrome.

The purified human biological fluid which can be obtained using the above method can also be used to prepare a composition intended for the therapeutic treatment of patients whose cells have undergone a stress, and preferably ultraviolet irradiation. The purified human biological fluid which can be obtained using the above method can also be used to prepare a composition intended for the therapeutic treatment of patients suffering from chronic infectious diseases having autoimmune manifestations, preferably selected from AIDS, hepatitis B and hepatitis C.

According to another aspect, the invention relates to a compound, characterized in that it is chosen from an antibody, an anti-idiotype antibody, a polypeptide, a polynucleotide, an antisense polynucleotide, an oligonucleotide, a vector, an antisense vector, a cell and a ligand according to the invention, as a medicinal product, and in particular as active principles of a medicinal product; these compounds will preferentially be in soluble form, combined with a pharmaceutically acceptable vehicle. The expression “pharmaceutically acceptable vehicle” is intended to denote any type of vehicle conventionally used in preparing injectable compositions, i.e. a diluent or a suspending agent such as an isotonic or buffered saline solution. Preferably, these compounds will be administered systemically, in particular intravenously, intramuscularly, intradermally or orally. Their optimal methods of administration, doses and pharmaceutical forms can be determined according to the criteria generally taken into account in establishing a treatment suitable for a patient, such as, for example, the age or body weight of the patient, the seriousness of his or her general condition, the tolerance to the treatment and the side effects observed, etc. When the agent is a polypeptide, an antagonist, a ligand, a polynucleotide, for example an antisense composition, or a vector, for example an antisense vector, it can be introduced into host cells or tissues in a certain number of ways, including viral infection, microinjection or vesicle fusion. It is also possible to use jet injection for intramuscular administration, as described by Furth et al. (1992). It is also possible to deposit the polynucleotide onto gold microparticles, and to deliver it intradermally using a particle bombardment device, or a “gene gun” as described in the literature (see, for example, Tang et al. (1992)), in which the gold microprojectiles are coated with the polynucleotide of the invention, preferably the antisense polynucleotide of the invention, and are then bombarded into skin cells.

More particularly, the compound as a medicinal product of the invention is intended for the prevention and/or treatment of autoimmune diseases preferably selected from the group composed of systemic lupus erythematosus and Sjögren's syndrome. The invention is also directed toward providing a pharmaceutical composition for the preventive and curative treatment of systemic lupus erythematosus and/or of Sjögren's syndrome, characterized in that it contains a therapeutically effective amount of a compound according to the invention and a pharmaceutically acceptable vehicle. This pharmaceutical composition may more particularly contain any antisense sequence or vector comprising such a sequence, or any inhibitor such as murabutide.

The Ro/SSA-like factor of the invention is a cellular protein which is capable of interacting with the CIS-acting elements of RNA viruses. Specifically, there are commonly sequences at the 5′ and 3′ ends of viral genomes which are important for viral replication. Among these sequences, mention should be made of the sequences involved in translation and/or in transcription of the viral genome; these sequences generally have the ability to form a stem loop with which the Ro/SSA-like protein of the invention is capable of interacting. Such interactions are easily demonstrated by those skilled in the art with RNA gel-shift experiments and/or with U.V. coupling experiments. The polypeptide of the invention is therefore capable of interacting with the CIS-acting elements of RNA viruses and thus of intervening in the RNA virus replication process. One of the objects of the present invention is therefore to provide compounds as medicinal products capable of inhibiting, impairing, and/or preventing the interactions of the polypeptide of the invention with sequences of the genome of an RNA virus which has infected patients' cells. Among these compounds, mention should be made more particularly of the anti-Ro/SSA-like antibodies and the Ro/SSA-like inhibitors and antagonists. It is also within the scope of the invention to use the ligands which can be obtained using the screening method of the invention, such as murabutide, to inhibit, impair and/or destroy the transcription of the gene naturally encoding the polypeptide of the invention, and also antisense polynucleotides or vectors to inhibit, impair and/or prevent, directly or indirectly, the interactions of the polypeptide of the invention with sequences of the genome of an RNA virus which has infected patients' cells. Among the RNA viruses the replication of which is liable to be affected by ligands or a compound of the invention, mention should be made nonexhaustively of (a) viruses of the family Togaviridae, and more particularly alphaviruses such as the Sinbis virus, flaviviruses such as the yellow fever virus, rubiviruses such as the rubella virus, pestiviruses; (b) Coronaviridae; (c) Retroviridae such as oncoviridae, spumaviridae and lentiviruses, and more particularly the human acquired immunodeficiency syndrome virus (HIV); (d) Paramyxoviridae such as the parainfluenza virus, the Sendai virus, the Newcastle disease virus, the measles virus or the mumps virus; (e) Orthomyxoviridae, such as human influenza viruses (A, B and C); (f) Rhabdoviridae, such as the rabies virus; (g) Bunyaviridae, such as human encephalitis viruses; (h) Arenaviridae, such as viruses responsible for hemorrhagic fevers in humans, for instance the Ebola virus, the Lassa fever virus, or the virus of the Tacaribe-Pichinde complex; (g) Picornaviridae, such as human polio viruses, rhinoviruses (cold viruses), cardioviruses (encephalomyocarditis virus, Mengo virus), or the hepatitis A virus. The invention therefore relates to a compound according to the invention, as a medicinal product intended for the prevention and/or treatment of diseases selected from the pathologies caused by an infection with an RNA virus, and also the pharmaceutical composition for the preventive and/or curative treatment of a viral pathology preferably selected from the pathologies caused by an infection with an RNA virus, characterized in that it contains a therapeutically effective amount of a compound according to the invention and a pharmaceutically acceptable vehicle. Among the compounds as medicinal products of the invention, the compounds which are antagonists of the Ro/SSA-like polypeptide are particularly preferred for preparing a medicinal product intended for the treatment of viral pathologies; among these antagonist compounds, mention should be made more particularly of the antisense polynucleotides and/or the antisense vectors.

The invention also relates to a compound according to the invention intended for the prevention and/or treatment of chronic infectious pathologies having autoimmune manifestations, such as AIDS or hepatitis B and C. Specifically, it has been demonstrated that some HIV+ patients are liable to develop high levels of immunoglobulins G (IgGs) which react with double-stranded DNA, with histone H2A-derived synthetic peptides having ubiquitin residues, with the Sm-D antigen, with the RNP U1-A antigen or with the 60 kD Ro/SSA antigen (Muller et al., 1992). The invention also relates to a pharmaceutical composition for the preventive and curative treatment of a chronic infectious disease having autoimmune manifestations, preferably selected from the group composes of AIDS, hepatitis B and hepatitis C characterized in that it contains a therapeutically effective amount of a compound according to the invention and a pharmaceutically acceptable vehicle.

More particularly, the present invention relates to the use of muramyl peptides, in particular murabutide, for preparing a medicinal product intended for the preventive and/or curative treatment of diseases selected from the group composed of autoimmune diseases, chronic infectious pathologies having autoimmune manifestations, and viral pathologies, with the exception of those caused by the human immunodeficiency virus (HIV).

According to another embodiment, it is also within the scope of the invention to provide a method of therapeutic or prophylactic treatment of a disease associated with an increase in the expression or in the activity of the Ro/SSA-like polypeptide according to the invention. This method comprises administering a therapeutically effective amount of an antagonist of the polypeptide of the invention to a patient who requires such a treatment.

More generally, the present invention relates to the use of a compound according to the invention, for preparing a medicinal product intended to neutralize the anti-Ro/SSA-like autoantibodies present in a patient's biological fluid. The invention also relates to the use of an antisense polynucleotide and/or of an antisense vector according to the invention, for preparing a medicinal product intended to decrease the expression of the Ro/SSA-like polypeptide of the invention. The invention also relates to the use of a compound according to the invention, for preparing a medicinal product intended for the treatment of infections with RNA viruses.

Other characteristics and advantages of the invention are apparent in the remainder of the description with the examples represented below. In these examples reference will be made to the following figures.

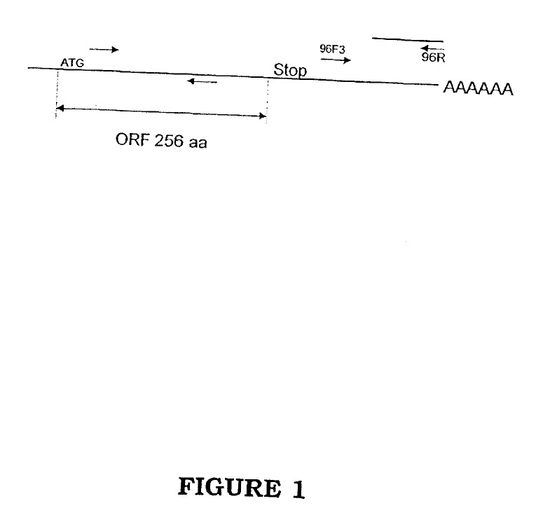

FIG. 1: Strategy used to obtain the corresponding cDNA for the Ro/SSA-like protein. The initial 152 bp fragment obtained by DD-RT-PCR corresponds to the upper line. The 3′ region of the cDNA was obtained after a 3′ RACE (Rapid Amplification of cDNA ends), the fragment obtained of approximately 2 800 bp corresponds to the lower line; this fragment has an open reading frame 256 amino acids long. A PCR (5′ RACE) reaction using the oligonucleotide 96F3 allowed the inventors to identify the poly A+ tail of the cDNA; this tail is located in the position 3′ of the fragment initially obtained by DD-RT-PCR.

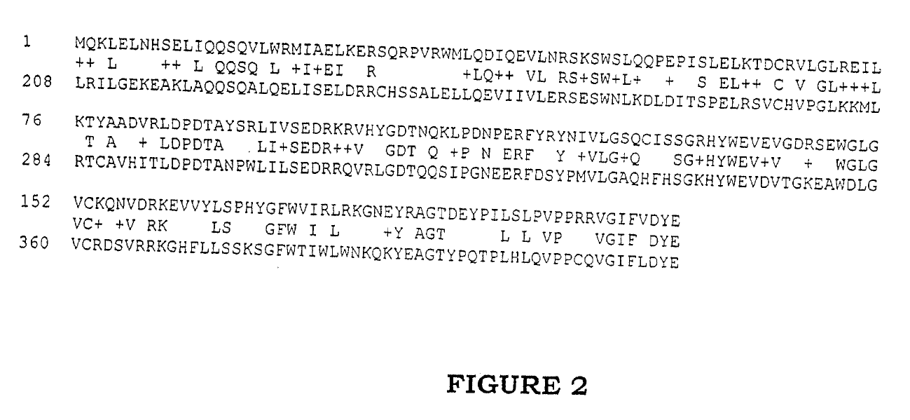

FIG. 2: Sequence homology of the 256 amino acid open reading frame with the 52 kDa human Ro/SSA protein. The upper line represents the 256 amino acid sequence of the open reading frame. The lower line corresponds to the sequence of the human 52 kDa Ro/SSA-like protein. The intermediate line indicates the homologous or equivalent amino acids.

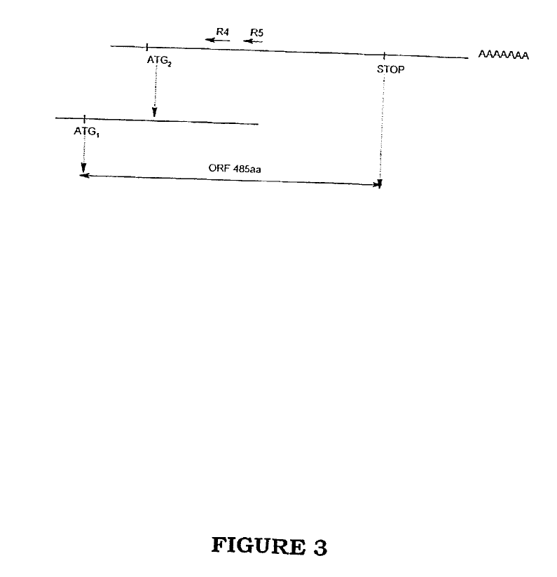

FIG. 3: Strategy used in order to obtain the 5′ region of the cDNA by virtue of a 5′ RACE approach using the primers 96R4 (SEQ ID No. 11) and 96R5 (SEQ ID No. 12). This approach made it possible to obtain a fragment of approximately 1 000 bp (lower line) containing a potential START (ATG) codon called ATG1.

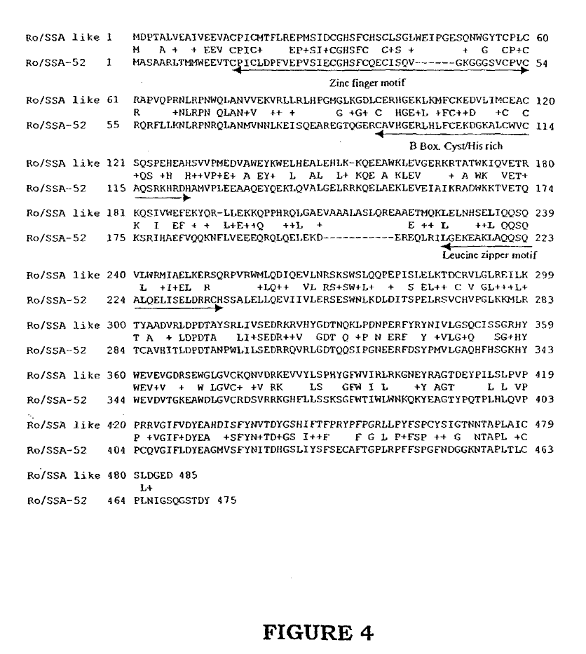

FIG. 4: Amino acid sequence alignment of the Ro/SSA-like protein (485 aa) with the 52 kDa Ro/SSA protein (475 aa). The 52 kDa Ro/SSA protein comprises several characteristic domains; region 16-54 bears the zinc finger motif, region 91-123 is a cysteine- and histidine-rich region (Cyst/His rich) called B Box, domain 190-245 bears the leucine zipper motif.

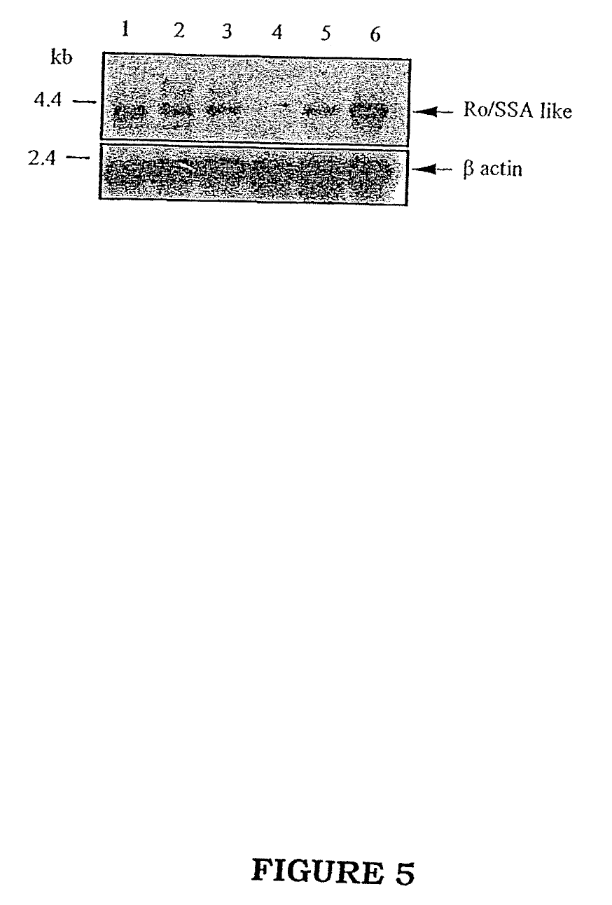

FIG. 5: Northern blotting analysis of the expression of the mRNA encoding RO/SSA-like. (1) Spleen, (2) lymph node, (3) thymus, (4) PBMCs, (5) bone marrow, (6) fetal liver. β-actin mRNA is used as an internal control.

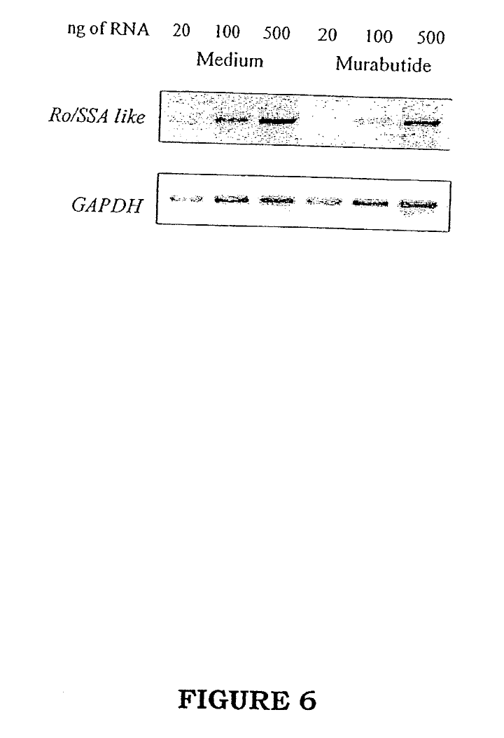

FIG. 6: Study by semiquantitative RT-PCR of the differential expression of the mRNA encoding the Ro/SSA-like protein in PBMCs of HIV+ patients. The study is carried out in the presence (“murabutide”) and absence (“medium”) of murabutide in the culture medium. The different amounts of matrix RNA were used. Expression of GAPDH is used as an internal control.

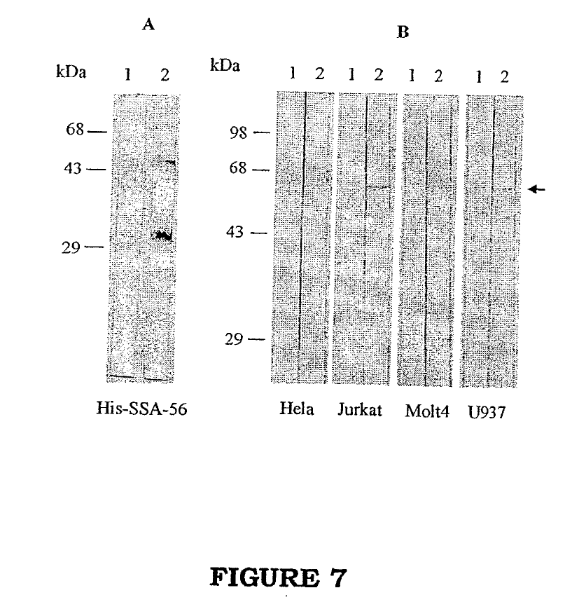

FIG. 7: Reactivity of the mouse anti-Ro/SSA-like antiserum (anti-SSA-56) against the recombinant protein (A) and various cell extracts (B). 1 μg of recombinant His-SSA-56 protein and 130 μg of cell extracts were used per lane. 1 represents the healthy mouse control and 2 represents the anti-SSA-56 antiserum diluted to 1/100 in A and to 1/50 in B.

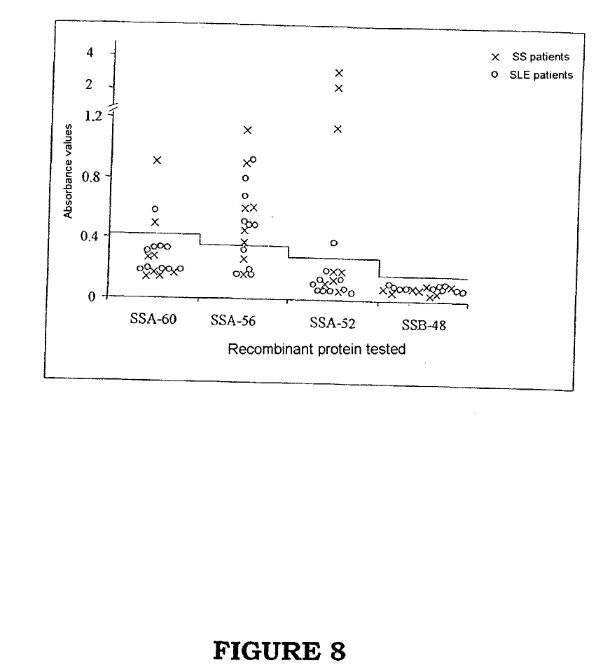

FIG. 8: Anti-SSA and anti-SSB autoantibody titer tested by ELISA in the sera of 10 SS patients, and 8 SLE patients who were found to be negative (SSA⁻ and SSB⁻) using the conventional Ouchterlony test. 1 μg/ml of each recombinant protein was marked in 96-well plates. The sera were diluted to 1/900. The horizontal bars indicate the thresholds of discrimination of positive activity, which correspond to the mean plus twice the standard deviation of the values obtained in the healthy controls.

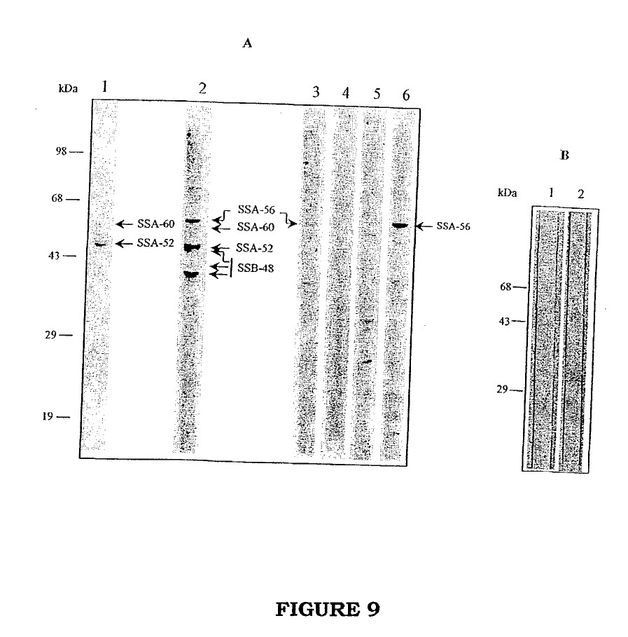

FIG. 9: Reactivity of patients' sera against Hela cells (A) and of the recombinant Ro/SSA-like protein (SSA-56) (B) tested by Western blotting. 150 μg of total extract were used per lane for A and 1 μg of His-SSA-56 for B. Lanes 1, 2 and 3 of (A) represent the sera diluted to 1/50 of 3 different patients and lane 4 represents the healthy control. Lane 5 corresponds to the healthy mouse serum diluted to 1/50 and lane 6 corresponds to the mouse anti-SSA-56 antiserum. Lane 1 of (B) corresponds to the healthy control and lane 2 corresponds to a patient's serum diluted to 1/20.

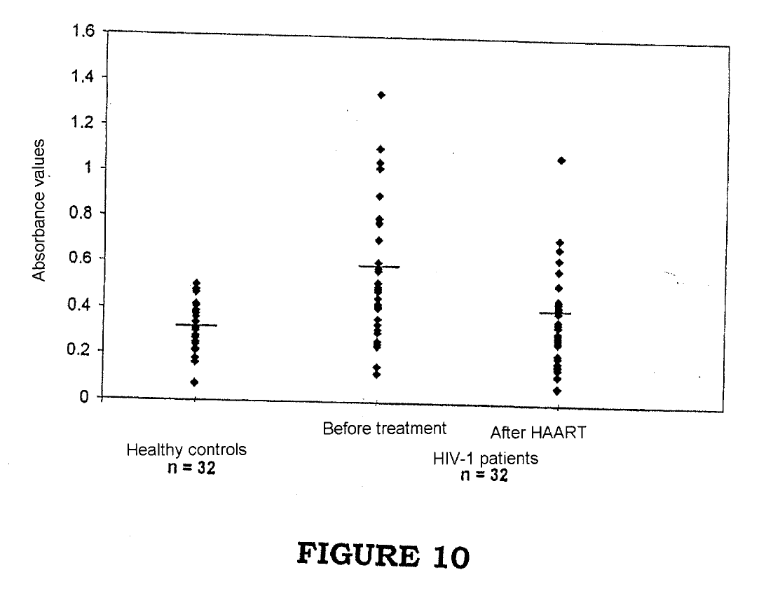

FIG. 10: Anti-SSA-56 autoantibody titer tested by ELISA in the sera of HIV+ patients tested at 1/300 before and after antiretroviral treatment (HAART).

### EXAMPLES

### Example 1

Strategy for Cloning the Novel Poly-Nucleotide Sequence Encoding Ro/SSA-Like

Differential-Display-RT-PCR (DD-RT-PCR) experiments were carried out using PBMCs from an HIV+ patient. The inventors selected more than 130 cDNA fragments differentially expressed after treatment of the PBMCs from an HIV+ patient with murabutide (MB). These fragments were subcloned into the vector Pcr2.1 (Invitrogen) and then sequenced by automatic sequencing (ABI Prism 377, Perkin-Elmer). The sequences were analyzed for homology searches using the databanks and the Basic Local Alignment Search Tool (Blast 2) server of the NCBI.

Based on a fragment 152 bp long (SEQ ID No. 5) obtained by DD-RT-PCR, the inventors synthesized two specific primers, including 96 R: 5′ TGC GTT TAT TTC TCC AGT TTG GCC TAT TTT AAC 3′ (SEQ ID No. 6), in order to carry out a first amplification by 5′ and 3′ RACE (for Rapid Amplification of cDNA Ends). The inventors were thus able to obtain a fragment of approximately 2 800 bp (SEQ ID No. 7) by virtue of the amplification using 96 R. This fragment was sequenced in several steps using the internal primers 396: 5′ GTG AGA AGT TTC AGA CCC AAA TAT 3′ (SEQ ID No. 9), 395: 5′ CCA GCC GAT TAC TAG TAG AGA AAA AGC 3′ (SEQ ID No. 10), 421: 5′ GCA TCT CGT CAG GCC GGC ACT ACT 3′ (SEQ ID No. 11), 420: 5′ CTT GCT CCC TTA AGG CCA TTT CAG 3′ (SEQ ID No. 12). The 2 800 bp sequence (SEQ ID No. 7) has an open reading frame (ORF) of 256 amino acids up to a potential Stop codon.

The inventors compared this ORF with sequences present in the databanks, and thus identified homologous proteins exhibiting a relative homology with this ORF. They also identified the presence of a domain of the leucine zipper type, consisting of a leucine-rich sequence of the (L-(X)₆-L-(X)₆-L-(X)₆) type. More particularly, the inventors observed that this ORF exhibits 46% identity with the human 52 kDa Ro/SSA protein; this ribonucleoprotein, the function of which remains unknown and which is composed of a single polypeptide and an RNA molecule, is located in the cytoplasm or in the nucleus and in many mammalian cells. At least two isoforms exist, which are encountered in different cell types and tissues. The particularity of this protein is its ability to bind an RNA molecule. Sera from patients suffering from systemic lupus erythematosus and from Sjögren's syndrome often have antibodies against the normal cellular protein. It should be noted that there is no zinc finger domain in this ORF, whereas this is present in the Ro/SSA protein.

In order to obtain the 3′ portion of the cDNA, the inventors synthesized the primer 96 F3: (5′ CCT GTC TGA GGC ATA GAG GCA GGC AAG CCG 3′) (SEQ ID No. 13), which made it possible to obtain a fragment of approximately 500 bp which made it possible to confirm the presence of the polyA+ tail 3′ of the fragment initially obtained by DD-RT-PCR.

FIG. 1 gives a diagrammatic representation of the strategy used; FIG. 2 gives the sequence homologies of the 256 amino acid open reading frame of the fragment of approximately 2 800 bp with the 52 kDa human Ro/SSA protein.

Since the sequence of approximately 2 800 bp (SEQ ID No. 7) did not correspond to that obtained after Northern blotting, the inventors therefore synthesized two nucleotide primers, based on this sequence, in order to carry out further 5′ RACE reactions. For this, a new matrix was synthesized and amplified from total RNA of HIV+ patient PBMCs not stimulated with MB. The PCRs carried out using the primers 96 R4 (5′-CCT GGC TCT GCT GGA TGA GCT CGC TAT-3′) (SEQ ID No. 14) and 96 R5 (5′-TCA ACT CTG CAA TCA TCC TCC ACA GGA-3′) (SEQ ID No. 15) revealed the presence of a fragment of approximately 1 000 bp which was cloned and sequenced (SEQ ID No. 16). The sequence shows that the two Stop codons initially obtained are not found again; in addition, the reading frame remains open and the amino acid sequence is again homologous to the 52 kDa Ro/SSA protein. The latter fragment has a potential ATG preceded by a STOP codon. The reading frame is now 485 aa. The strategy used in order to obtain the 5′ region of the cDNA by virtue of a 5′ RACE approach using primers 96 R4 and R5 is given in FIG. 3.

A 5′ RACE reaction was carried out using a new primer, 96R6 (5′-TCA CCC TTC AGC CCC ATT CCT GGA TGT-3′) (SEQ ID No. 17), in order to confirm the presence of the ATG₁ and of the Stop codon before this (FIG. 3). PCR made it possible to amplify a fragment of approximately 550 bp, and this fragment was cloned and sequenced. It made it possible to confirm the presence of ATG. The amino acid sequence alignment of the new 485 amino acid reading frame with the 52 kDa Ro/SSA protein is given in FIG. 4.

A PCR was carried out using specific primers containing the START1 codon and the STOP codon, on an RT (Reverse Transcripts of total RNA) of PBMCs, in order to amplify the copy of the cDNA encoding the Ro/SSA-like protein. The inventors obtained a fragment of expected size, and this fragment was cloned and sequenced. The complete nucleic acid sequence corresponds to the sequence SEQ ID No. 1. The open reading frame is 485 aa (SEQ ID No. 2).

The nucleic acid sequence (SEQ ID No. 1) was compared with various databanks; it is partially homologous to the clone NT2RM2001575 (homo sapiens cDNA FJ10369 fis) bearing the accession number AK001231 (SEQ ID No. 3) for the DDBJ/EMBL/GenBank databank. No study of the biological activity of the corresponding protein has been carried out; the deduced amino acid sequence (SEQ ID No. 4) corresponds partially to the sequence SEQ ID No. 2.

### Example 2

Study of the Expression of the Corresponding mRNA

2.1. Study by Northern Blotting of the Expression of the mRNA in Various Tissues

The fragment corresponding to the 256 amino acid open reading frame was used as a probe and was labeled with ³²P (Megaprime, Amersham). Hybridization of a membrane containing 2 μg of poly A+ RNA (Clontech) originating from spleen (1), from lymph node (2), from thymus (3), from PBMCs (4), from bone marrow (5) and from fetal liver (6) revealed the result given in FIG. 5.

It is noted that expression of the corresponding mRNA is represented weakly in the PBMCs compared with other lymphoid tissues.

2.2. Study by Semiquantitative RT-PCR of the Expression of the mRNA in PBMCs from HIV+ Patients or from Healthy Controls

2.2.1 Isolation and Treatment of PBMCs:

PBMCs from patients (P) infected with HIV or from control healthy donors (C) are isolated, depleted of CD8+ (Dynabeads, Dynal) and stimulated with PHA (5 μg/ml) for 3 days. The cells are then treated or not treated with murabutide (10 μ/ml) in the presence of interleukin 2 (IL2) (10U/ml) in RPMI medium supplemented with 10% fetal calf serum (FCS) for 6 hours or 24 hours in a proportion of a minimum of 5×10⁶ cells per condition.

2.2.2 RT-PCR:

After treatment, the RNA in the cells is extracted (RNAplus, Quantum-bioprobe), then treated with DNase (Boerhinger) and reverse transcribed (RT) using an oligo(dT) in the presence of the Mu-MLV reverse transcriptase (Superscript II, Gibco).

The quality of the RTs is verified by PCR (25 cycles) with primers specific for GAPDH (5′GCC ATC AAT GAC CCC TTC ATT GAC 3′) (SEQ ID No. 18) and (5′TGA CGA ACA TGG GGG CAT CAG CAG 3′) (SEQ ID No. 19) using 20, 100 and 500 ng of total RNA. The inventors then carried out RT-PCRs (35 cycles) with primers specific for the mRNA encoding Ro/SSA-like, (5′ GAA AGA GAG GTC GCA GAG GCC TGT 3′) (SEQ ID No. 20) and (5′ TGA TAA GGC TGA GGA AGG GAA ATG 3′) (SEQ ID No. 21). The number of amplification cycles was determined beforehand (35 cycles). The amplified fragments are visualized on agarose gel (1%) in the presence of ethidium bromide, and then quantified using the Imager master program (Pharmacia).

2.2.3 Evaluation of the Differential Expression:

For each dilution, the given value for the gene studied is related to that of the GAPDH (ratio=R). For each patient and each time (6 h or 24 h), the R of the cells treated with murabutide is related to that of the untreated cells. The results are then expressed as % increase or inhibition of expression of the gene relative to the untreated cells. It should be noted that, for each dilution tested, the R can vary slightly; the mean for the Rs was produced by taking care to always be in the linear phase of amplification.

2.2.4 Results:

The inventors tested the differential expression of the mRNA encoding the Ro/SSA-like protein on 10 HIV+ patients and 8 healthy controls. The results revealed a significant inhibition of the expression of the mRNA in the PBMCs from HIV+ patients after stimulation with murabutide (7 patients/10 show an inhibition of more than 40%), whereas, in the PBMCs from healthy controls, the inhibition is weaker and was observed only in 3 cases out of 8. FIG. 6 illustrates the inhibition observed in a patient.

### Example 3

Expression of Recombinant Proteins in an E. Coli Bacterial System (pQE)

3.1. Expression of a Partial Recombinant Protein Corresponding to the 256 aa Reading Frame

A PCR was carried out on the cDNA of PBMCs using nucleotide primers corresponding to the ATG₂ (5′-GCA GCC CGG GCC ATG CAG AAA CTG GAG TTG-3′) (SEQ ID No. 22) and to the STOP (5′-GGT GGT CTG CAG CTT AGT CCT CCC CAT CCA-3′) (SEQ ID No. 23). These primers contain, respectively, the restriction sites corresponding to the Sma I and Pst I restriction enzymes. The fragment obtained was cloned into the vector pCR2.1 (Invitrogen).

The insert present in the vector pCR2.1 (Invitrogen) was excised from the vector with the Sac I enzyme (site located 11 aa from the ATG₂) and Pst I enzyme (Stop) and then inserted into the vector pQE30 (Sac I/PstI).

After transformation of M15 bacteria, the expression of the recombinant protein was induced with IPTG for 5 hours and then purified on nickel beads. This purification, carried out under denaturing conditions, made it possible to obtain a recombinant Ro/SSA-like protein resolubilized in the presence of 0.1% SDS.

3.2. Production of an Antiserum against the Recombinant Protein

Two batches of 5 mice were immunized with 50 μg or 100 μg of resolubilized protein, in the presence of complete Freund's adjuvant, and a second immunization took place three weeks later in the presence of incomplete adjuvant.

The mice were bled three weeks after the first immunization (S1) and one week after the second immunization (S2). The sera were tested by ELISA on the purified recombinant protein. The sera giving high titers were used in experiments to detect the presence, on membranes obtained by Western blotting, of the recombinant protein and also the native protein present in total antigen extracts from PBMCs of HIV+ patients. Three sera were tested on antigen extracts from PBMCs of two different patients.

3.3. Expression of the Complete Recombinant Protein Corresponding to the 485 aa Open Reading Frame

A PCR was carried out on the cDNA of PBMCs using nucleotide primers corresponding to the ATG₁ (5′-TGA GAA GCA TGC ATG GAT CCC ACA GCC TTG-3′) (SEQ ID No. 24) and to the STOP (5′-GTG GTA CCC GGG TTA GTC CTC CCC ATC CAG-3′) (SEQ ID No. 25). The fragment was cloned into the vector pCR2.1 and then sequenced.

The fragment was digested with the Sph 1 and Sma 1 enzymes and then inserted into the vector pQ80. Purification was carried out according to the points described in § 3.1. The inventors obtained the recombinant protein with the expected size of 58 kDa and also two degradation products 51 and 34 kDa in size.

The protein thus expressed was used to immunize mice according to the conditions described in paragraph 3.2.

### Example 4

Expression of the Recombinant Protein in a Eukaryotic System

Since the homologous Ro/SSA protein is expressed in the cytoplasm or in the nucleus of mammalian cells, the inventors developed a strategy of overexpression of the recombinant protein in eukaryotic cells in order to assess its role in regulating HIV.

For this, the complete copy of the cDNA was amplified using the primers 96 GFP Xho I (5′-GTG TGA CTC GAG ACC ATG GAT CCC ACA GCC-3′) (SEQ ID No. 26) and 96 GFP Eco RI (5′-CCG GAA TTC CGT CCT CCC CAT CCA GGG A-3′) (SEQ ID No. 27); this was cloned into the vector pEGFP digested with Xho I/Eco RI, and then sequenced. The cDNA encoding GFP is 3′ of the cloned insert.

In addition, the complete copy of the cDNA was amplified using the primers 96 His RI (5′-CCG GAA TTC ACC ATG GAT CCC ACA GCC-3′) (SEQ ID No. 28) and 96 His Xho I (5′-GCT TTC CTC GAG GTC CTC CCC ATC CAG GGA-3′) (SEQ ID No. 29); this was cloned into the vector pcDNA₆ digested with Eco RI/Xho I. In this system, the protein is fused to 6 histidines and to a V5 protein.

The complete recombinant protein was used to immunize mice and made it possible to obtain an antiserum. This serum, in Western blotting, recognizes the complete recombinant protein and also the degradation products (FIG. 7a). The antiserum recognizes the corresponding native protein in protein extracts of Hela, Jurkat, Molt4 and U937 cells, with a size of approximately 63 kDa (FIG. 7B). The cellular localization of the native protein was carried out by immunofluorescence on Hela cells using the mouse antiserum. The protein is located in the cytoplasm and more strongly around the nucleus (perinuclear).

### Example 5

Production of a Monoclonal Antibody against the Recombinant Protein

Hybridomas were obtained after fusion of spleen cells, originating from the mice immunized with the recombinant protein, with SP20 myeloma cells. After selection according to the method of Köhler and Milstein (1976), the presence of antibodies against the recombinant protein is detected by ELISA in the hybridoma supernatants. The positive hybridomas are then cloned by limiting dilution in order to obtain a single cell secreting monoclonal antibodies against a single epitope. The supernatants are tested by ELISA and by Western blotting. A positive clone which recognizes the whole recombinant protein and also the degradation products was selected. The specificity of this clone, JE5, is verified by ELISA against other antigens produced in the same bacterial expression system as the Ro/SSA-like (SSA-56) protein, and is summarized in table I below. It should be noted that the anti-SSA-56 monoclonal antibody JE5 does not recognize the other proteins belonging to the SS autoantigen family (SSA-52, 60 and SSB-48).

TABLE I

Absorbance value at 1/100

| | | |
|---|---|---|
| | Monoclonal | Control |
| Proteins tested | JE5 (anti-SSA-56) | SP20 |

| | | |
|---|---|---|
| SSA-56 | 2.3 | 0.058 |
| SSA-52 | 0.082 | 0.059 |
| SSA-60 | 0.074 | 0.068 |
| SSB-48 | 0.074 | 0.051 |
| Helicase | 0.093 | 0.055 |
| HIV Tat | 0.078 | 0.068 |
| HIV Nef | 0.075 | 0.056 |

### Example 6

Demonstration of Anti-RoSSA-like Autoantibodies in Sera of Patients Suffering from Sjögren's Syndrome

6.1 Analysis by ELISA

In order to analyze whether the novel Ro/SSA-like (SSA-56) protein may be a target for the autoantibodies present in certain autoimmune diseases such as Sjögren's syndrome, the inventors tested the sera of 6 patients in order to study the presence of antibodies against the Ro/SSA-like protein of the invention. The inventors also analyzed the sera of 5 healthy donors who had no symptom of the disease.

The patients were analyzed, in the hospital laboratory, as having or not having anti-SSA or anti-SSB or anti-SSA and anti-SSB antibodies. The analyses were done by ELISA; to do this, the wells of 96-well plates are covered with the Ro/SSA-like protein of the invention, the sera are incubated at various dilutions and the presence of the antibodies is revealed with an anti-human IgG antibody conjugated with peroxidase. The enzymatic activity is revealed with the peroxidase substrate (O-phenylenediamine) and the absorbance values for each well are obtained after reading on an ELISA plate spectrophotometer. The results are shown in table II below:

TABLE II

| | |
|---|---|
| | Anti-Ro/SSA-like autoantibody |
| Presence of | titer measured as absorbance |

| Individual | anti-SSA/SSB | value in the sera diluted | | |
|---|---|---|---|---|
| tested | antibodies | 1/500 | 1/100 | |
| Healthy | | | | |
| donors | | | | |
| 1 | − | 0.12 | 0.22 | (−) |
| 2 | − | 0.10 | 0.17 | (−) |
| 3 | − | 0.10 | 0.19 | (−) |
| 4 | − | 0.19 | 0.30 | (−) |
| 5 | − | 0.18 | 0.30 | (−) |
| Sjögren's | | | | |
| syndrome | | | | |
| 1 | SSA-/SSB− | 0.14 | 0.23 | (−) |
| 2 | SSA-/SSB− | 0.51 | 1.00 | (+) |
| 3 | SSA+/SSB− | 0.11 | 0.17 | (−) |
| 4 | SSA+/SSB− | 0.21 | 0.38 | (+) |
| 5 | SSA−/SSB+ | 0.41 | 0.51 | (+) |
| 6 | SSA+/SSB+ | 0.37 | 0.63 | (+) |

These results clearly show the presence of autoantibodies against the Ro/SSA-like protein in the patients suffering from Sjögren's syndrome (4 out of 6 patients). The most interesting observation is that patient 2, who does not have anti-SSA and anti-SSB antibodies, is the most positive against the novel Ro/SSA-like protein of the invention. This suggests that the novel protein may be of considerable value in confirming the diagnosis of the disease, and confirms the discovery of a new member of the SS antigen family.

The preceding analysis, carried out on 6 SS patients, was completed by analyzing 25 SS patients, 22 SLE patients and 25 healthy controls. Each patient serum was tested by ELISA against the recombinant SSA-52, SSA-60, SSB-48 and SSA-56 proteins (table III). The results show the presence of anti-SSA-56 (anti-Ro/SSA-like) autoantibodies in the SS and SLE patients who may or may not have autoantibodies against the SSA-52, SSA-60 and SSB-48 proteins.

In order to show the importance of the presence of the anti-SSA-56 autoantibodies in the patients' sera for diagnostic purposes, the inventors selected the SS and SLE patients given negatives by the conventional Ouchterlony method for diagnosis. Out of 18 patients selected, none is positive for SSB-48, 4 are positive for SSA-52, and 3 are positive for SSA-60. Among these patients, 12 are positive for SSA-56, which shows that patients who are negative for the other SSAs and SSBs have anti-SSA-56 autoantibodies (FIG. 8). These results show the importance of the Ro/SSA-like (SSA-56) protein in confirming the diagnosis of patients suffering from SS or from SLE.

TABLE III
Titer of antibodies against the recombinant SSA and SSB
proteins in the serum of healthy controls, of SS
patients and of SLE patients

| | Healthy | | |
|---|---|---|---|
| | controls | SS patients | SLE patients |
| Antigen | (n = 25) | (n = 25) | (n = 22) |
| tested | Mean absorbance values* | | |
| SSA-56 | 0.15 | 0.45§ | 0.46§ |
| (Ro/SSA-like) | (0.19 ± 0.02)‡ | (0.59 ± 0.11) | (0.49 ± 0.06) |
| SSA-52 | 0.15 | 0.26§ | 0.20 |
| | (0.14 ± 0.01) | (1.11 ± 0.25) | (1.16 ± 0.28) |
| SSA-60 | 0.16 | 0.29§ | 0.36§ |
| | (0.20 ± 0.02) | (0.66 ± 0.19) | (1.12 ± 0.23) |
| SSB-48 | 0.07 | 0.07 | |
| | (0.08 ± 0.01) | (0.38 ± 0.15) | (0.51 ± 0.21) |

*Tested with a 1/900 dilution of the serum
‡ Mean ± standard deviation
§ Significantly different from the values corresponding to the healthy controls (p < 0.05);
Mann Whitney U Rank test

6.2 Analysis by Western Blotting

The inventors tested the reactivity of a patient's serum against the recombinant protein by Western blotting (FIG. 9B). This serum recognizes the various fragments of the recombinant protein. In order to verify whether the patients' sera recognize the native protein, the inventors performed Western blotting on an extract of Hela cells (FIG. 9A). Patient No. 1 recognizes the native SSA-52 protein and more weakly SSA-60. Patient No. 2 recognizes SSA-52, SSA-60, SSB-48 and SSA-56, whereas patient No. 3 recognizes only the SSA-56 protein. Patient No. 4 corresponds to a healthy control. The size of the native protein is determined as a function of the size of the protein recognized by the anti-SSA-56 mouse polyclonal (lane 6).

### Example 7

Demonstration of Anti-Ro/SSA-Like (Anti-SSA-56) Autoantibodies in the Sera of HIV Patients before and after Antiretroviral Treatment (HAART)

The presence of anti-Ro/SSA autoantibodies was demonstrated in chronic viral diseases having clinical manifestations similar to autoimmune diseases, such as AIDS. The inventors analyzed the presence of anti-SSA-56 (anti-Ro/SSA-like) autoantibodies in the sera of HIV+ patients before and after treatment with HAART (FIG. 10). 32 healthy controls and 32 HIV+ patients were analyzed by ELISA against the recombinant SSA-56 protein. The results show the presence of SSA-56 autoantibodies in the HIV+ patients before treatment, and that the titer for these antibodies decreases significantly after the antiretroviral treatment. This indicates a hyperactivation of the immune system in HIV+ patients, and that the Ro/SSA-like (SSA-56) protein is a target (among others) of the autoimmune reaction against the host's proteins in patients clinically infected with HIV.

### REFERENCES

Buckholz (1993), Curr. Op. Biotechnology 4, 538.

Carter (1993), Curr. Op. Biotechnology 3, 533.

Duck et al. (1990), Biotechniques, 9, 142.

Edwards and Aruffo (1993), Curr. Op. Biotechnology, 4, 558.

Epstein (1992), Médecine/Sciences, 8, 902.

Furth et al. (1992), Anal. Biochem. 205: 365.

Gorbalenya et al., 1988, Nature, 333: 22.

Guatelli et al. (1990), Proc. Natl. Acad. Sci. USA 87: 1874.

Kievitis et al. (1991), J. Virol. Methods, 35, 273.

Köhler and Milstein (1975), Nature, 256, 495.

Köhler and Milstein (1976), Eur. J. Immunol. 6: 511.

Kwoh et al. (1989), Proc. Natl. Acad. Sci. USA, 86, 1173.

Landegren et al. (1988), Science 241, 1077.

Linder et al., 1989, Nature, 337: 121-122.

Lopez-Luna et al. (1995), Scand. J. Rhumatol. 24: 293-299.

Luckow (1993), Curr. Op. Biotechnology, 4, 564.

Luking et al. (1998), 33: 259-296.

Matthews et al. (1988), Anal. Biochem., 169, 1-25.

Miele et al. (1983), J. Mol. Biol., 171, 281.

Muller et al. (1992), Aids, 6: 933.

Neddleman and Wunsch (1970), J. Mol. Biol. 48: 443.

Olins and Lee (1993), Curr. Op. Biotechnology, 4: 520.

Pause and Sonenbery, 1992, EMBO J. 11: 2643-2654.

Pearson and Lipman (1988), Proc. Natl. Acad. Sci. USA, 85: 2444.

Perricaudet et al. (1992), La Recherche, 23: 471.

Rader et al. (1989), J. Clim. Invest. 83: 1293-1298.

Rohlmann et al. (1996), Nature Biotech. 14: 1562.

Rolfs, A. et al. (1991), Berlin: Springer-Verlag.

Schartw et al., Fundamental Immunology, Second Edition, Paul W. E. Edition Raven Press, New York (1984), pp. 816-866.

Segev (1992), Kessler C. Springer Verlag, Berlin, New York, 197-205.

Sibilia (1998), Rev. Rhum. 65: 45-57.

Smith and Waterman (1981), Ad. App. Math. 2: 482.

Stewart and Yound (1984), Solid phase peptides synthesis, Pierce Chem. Company, Rockford.

Tang et al. (1992), Nature, 356: 152.

Temin (1986), Retrovirus vectors for gene transfer. In Kucherlapati R., ed. Gene Transfer.

Walker et al. 1982, EMBO J. 1:945-951.

1. An isolated polypeptide, named Ro/SSA-like, of amino acid sequence SEQ ID No. 2.

2. An isolated polypeptide, characterized in that it comprises a polypeptide chosen from:
a) a polypeptide of sequence SEQ ID No. 2;
b) a variant polypeptide of a polypeptide of amino acid sequences defined in a);
c) a polypeptide homologous to the polypeptide defined in a) or b) and comprising at least 80% identity with said polypeptide of a);
d) a fragment of at least 15 consecutive amino acids of a polypeptide defined in a), b) or c), with the exception of the fragment of sequence SEQ ID No. 4;
e) a biologically active fragment of a polypeptide defined in a), b) or c), with the exception of the fragment of sequence SEQ ID No. 4.

3. The polypeptide as claimed in either one of claims 1 and 2, characterized in that it comprises at least one conserved nucleic acid binding domain selected from the group composed of a “zinc finger” domain and a “leucine zipper” domain.

4. A purified or isolated polynucleotide, characterized in that it encodes a polypeptide as claimed in one of claims 1 to 3.

5. The polynucleotide as claimed in claim 4, as sequence SEQ ID No. 1.

6. An isolated polynucleotide, characterized in that it comprises a polynucleotide chosen from:
a) a polynucleotide of sequence SEQ ID No. 1;
b) a fragment of at least 15 consecutive nucleotides of the sequence SEQ ID No. 1, with the exception of the polynucleotide of sequence SEQ ID No. 3, of the polynucleotide of sequence SEQ ID No. 5505 of application EP 0 679 716, and of the polynucleotides of sequences AK001231 and N46696 of the EMBL databank;
c) a nucleic acid sequence exhibiting a percentage identity of at least 85%, after optimal alignment, with a sequence defined in a) or b);
d) the complementary sequence or the RNA sequence corresponding to a sequence as defined in a), b) or c).

7. The use of a polynucleotide as claimed in claim 6, as a primer for amplifying or polymerizing nucleic acid sequences.

8. The use, in vitro, of a polynucleotide as claimed in claim 6, as a probe for detecting nucleic acid sequences.

9. The use, in vitro, of a polynucleotide as claimed in claim 6, as a sense or antisense nucleic acid sequence for controlling the expression of the corresponding protein product.

10. The use of a polynucleotide as claimed in any one of claims 7, 8 and 9, characterized in that said polynucleotide is directly or indirectly labeled with a radioactive compound or a nonradioactive compound.

11. A recombinant cloning and/or expression vector comprising a polynucleotide as claimed in one of claims 4 to 6 or encoding a polypeptide as claimed in any one of claims 1 to 3.

12. A recombinant antisense expression vector comprising a polynucleotide as claimed in one of claims 4 to 6, characterized in that said polynucleotide is inserted in the reverse orientation in said vector.

13. A host cell, characterized in that it is transformed with a vector as claimed in either of claims 11 and 12.

14. An animal, except a human, characterized in that it comprises a cell as claimed in claim 13.

15. A method for preparing a recombinant polypeptide, characterized in that a host cell as claimed in claim 13 is cultured under conditions which allow the expression and, optionally, the secretion of said recombinant polypeptide, and in that said recombinant polypeptide is recovered.

16. A recombinant polypeptide obtained using a method as claimed in claim 15.

17. An isolated monoclonal or polyclonal antibody, and its fragments, characterized in that it selectively binds a polypeptide as claimed in one of claims 1 to 3 or 16.

18. An anti-idiotypic antibody, and its fragments, characterized in that it is against the antibody as claimed in claim 17.

19. A method for detecting and/or assaying a polypeptide as claimed in one of claims 1 to 3 or 16, in a biological sample, characterized in that it comprises the following steps:
a) bringing the biological sample into contact with an antibody as claimed in claim 17;
b) demonstrating the antigen-antibody complex formed.

20. A kit of reagents for carrying out a method as claimed in claim 19, in a biological sample, by immunoreaction, characterized in that it comprises the following elements:
a) a monoclonal or polyclonal antibody as claimed in claim 17;
b) where appropriate, the reagents for constituting the medium suitable for the immunoreaction;
c) where appropriate, the reagents for detecting the antigen-antibody complex produced during the immunoreaction.

21. A method for detecting and/or assaying a polynucleotide as claimed in any one of claims 4 to 6, in a biological sample, characterized in that it comprises the following steps:
a) isolating the DNA from the biological sample to be analyzed, or obtaining a cDNA from the RNA of the biological sample;
b) specifically amplifying the DNA using a polynucleotide as claimed in claim 7, used as a primer;
c) analyzing the amplification products.

22. A method for detecting and/or assaying a polynucleotide as claimed in any one of claims 4 to 6, in a biological sample, characterized in that it comprises the following steps:
a) bringing a polynucleotide as claimed in one of claims 4 to 6 into contact with a biological sample;
b) detecting and/or assaying the hybrid formed between said polynucleotide and nucleic acid of the biological sample.

23. A kit of reagents for carrying out a method as claimed in claim 21 or 22, characterized in that it comprises at least one polynucleotide as claimed in one of claims 4 to 6.

24. A DNA chip, characterized in that it contains a polynucleotide as claimed in one of claims 4 to 6.

25. A protein chip, characterized in that it contains a polypeptide as claimed in one of claims 1 to 3 or 16, or an antibody as claimed in claim 17, or an anti-idiotypic antibody as claimed in claim 18.

26. A method for screening ligands which affect the transcription in vitro and/or in vivo of the gene naturally encoding the polypeptide as claimed in claims 1 to 3, and which comprises the following steps:
a) bringing a cell chosen from the host cell of claim 13 and a eukaryotic cell, preferably a human cell, expressing the polypeptide as claimed in claims 1 to 3, into contact with one or more potential ligands, in the presence of reagents required to carry out a transcription reaction;
b) detecting and/or measuring the transcriptional activity.

27. A ligand obtained by the method as claimed in claim 26.

28. The ligand as claimed in claim 27, characterized in that it is murabutide.

29. An agent for diagnosing human autoimmune diseases, characterized in that said diagnostic agent is selected from:
a) a polypeptide as claimed in claims 1 to 3;
b) an anti-idiotypic antibody as claimed in claim 18;
c) a cell as claimed in claim 13 transformed with an expression vector as claimed in claim 11 capable of effectively expressing said polypeptide as claimed in claims 1 to 3.

30. The diagnostic agent as claimed in claim 29, characterized in that said polypeptide, said anti-idiotypic antibody and said anti-idiotypic antibody fragments are coupled to a solid support directly or indirectly via a spacer arm.

31. The diagnostic agent as claimed in claims 29 and 30, characterized in that said polypeptide, said anti-idiotypic antibody and said anti-idiotypic antibody fragments are labeled directly or indirectly with a signal-generating label.

32. An in vitro method for detecting anti-Ro/SSA-like autoantibodies in a human biological fluid, comprising the steps of:
a) bringing said biological fluid into contact with a diagnostic agent as claimed in claims 29 to 31, characterized in that said autoantibodies react with said diagnostic agent;
b) demonstrating the autoantibody/polypeptide complex or the autoantibody/anti-idiotype antibody complex formed.

33. The method as claimed in claim 32, characterized in that said autoantibodies are present in the biological fluid of a patient suffering from pathologies selected from the group composed of autoimmune diseases, chronic infectious pathologies having autoimmune manifestations, and viral pathologies.

34. The method as claimed in claim 33, characterized in that said autoimmune diseases are preferably chosen from systemic lupus erythematosus (SLE) and Sjögren's syndrome.

35. The method as claimed in claim 33, characterized in that said chronic infectious pathologies having autoimmune manifestations are preferably chosen from AIDS, hepatitis B and hepatitis C.

36. The method as claimed in claim 33, characterized in that said viral pathologies are preferably chosen from those caused by infection with an RNA virus.

37. The method as claimed in claim 32, characterized in that said autoantibodies are present in the biological fluid of a patient whose cells have undergone a stress, preferably ultraviolet irradiation.

38. A diagnostic kit, characterized in that it contains a diagnostic agent as claimed in any one of claims 29 to 31.

39. An in vitro method for purifying a human biological fluid liable to contain anti-Ro/SSA-like autoantibodies, comprising the steps of:
a) bringing said biological fluid into contact with a polypeptide as claimed in claims 1 to 3, or an anti-idiotypic antibody, or one of its fragments, as claimed in claim 18, under conditions which allow the formation of an autoantibody/polypeptide complex or of an autoantibody/anti-idiotype antibody complex formed;
b) separating the biological fluid and the complex formed in step a);
c) recovering the biological fluid obtained in step b).

40. The use of a purified human biological fluid obtained using the method as claimed in claim 39, for preparing a composition intended for the therapeutic treatment of patients suffering from autoimmune diseases preferably selected from the group of systemic lupus erythematosus (SLE) and Sjögren's syndrome.

41. The use of a purified human biological fluid obtained using the method as claimed in claim 39, for preparing a composition intended for the therapeutic treatment of patients whose cells have undergone a stress.

42. The use of a purified human biological fluid obtained using the method as claimed in claim 39, for preparing a composition intended for the therapeutic treatment of patients suffering from chronic infectious diseases having autoimmune manifestations, preferably selected from AIDS, hepatitis B and hepatitis C.

43. A compound, characterized in that it is chosen from:
a) a polypeptide as claimed in one of claims 1 to 3 or 16;
b) a polynucleotide as claimed in one of claims 4 to 6;
c) a polynucleotide as claimed in one of claims 4 to 6, used as an antisense nucleic acid sequence;
d) a vector as claimed in claim 11 or 12;
e) a cell as claimed in claim 13;
f) an antibody as claimed in claim 17;
g) an anti-idiotypic antibody as claimed in claim 18;
h) a ligand as claimed in claims 27 and 28, with the exception of muramyl peptides, and in particular of murabutide;
as a medicinal product.

44. The compound as claimed in claim 43, as a medicinal product intended for the prevention and/or treatment of diseases selected from the group composed of autoimmune diseases, chronic infectious pathologies having autoimmune manifestations, and viral pathologies.

45. The compound as claimed in claim 44, characterized in that said autoimmune disease is selected from the group composed of systemic lupus erythematosus and Sjögren's syndrome.

46. A pharmaceutical composition for the preventive and/or curative treatment of systemic lupus erythematosus and/or of Sjögren's syndrome, characterized in that it contains a therapeutically effective amount of a compound as claimed in claim 45 and a pharmaceutically acceptable vehicle.

47. The compound as claimed in claim 44, characterized in that said viral pathology is selected from the pathologies caused by infection with an RNA virus.

48. A pharmaceutical composition for the preventive and/or curative treatment of a viral pathology preferably selected from the pathologies caused by infection with an RNA virus, characterized in that it contains a therapeutically effective amount of a compound as claimed in claim 47 and a pharmaceutically acceptable vehicle.

49. A compound as claimed in claim 44, characterized in that said chronic infectious pathology having autoimmune manifestations is selected from the group composed of AIDS, hepatitis B and hepatitis C.

50. A pharmaceutical composition for the preventive and curative treatment of a chronic infectious disease having autoimmune manifestations, preferably selected from the group composed of AIDS, hepatitis B and hepatitis C, characterized in that it contains a therapeutically effective amount of a compound as claimed in claim 49 and a pharmaceutically acceptable vehicle.

51. The use of a compound as claimed in claim 43, for preparing a medicinal product intended to neutralize the anti-Ro/SSA-like autoantibodies in a biological fluid.

52. The use of a compound as claimed in claim 43, for preparing a medicinal product intended for the treatment of infections with RNA viruses.

53. The use of muramyl peptides, in particular of murabutide, for preparing a medicinal product intended for the preventive and/or curative treatment of diseases selected from the group composed of autoimmune diseases, chronic infectious pathologies having autoimmune manifestations, and viral pathologies, with the exception of those caused by the human immunodeficiency virus.
